(19) United States
(12) Patent Application Publication
(10) Pub. No.: US 2007/0129776 A1
(43) Pub. Date: Jun. 7, 2007

(54) External wearable light therapy treatment systems

(75) Inventors: Anthony J. Robins, Bellevue, WA (US); Phillip Burwell, Snohomish, WA (US); David B. Shine, Sammamish, WA (US); James C. Chen, Bellevue, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Light Sciences LLC, Snoqualmie, WA (US)

(21) Appl. No.: 11/584,345

(22) Filed: Oct. 20, 2006

Related U.S. Application Data
(60) Provisional application No. 60/728,556, filed on Oct. 20, 2005.

(51) Int. Cl. A61N 5/06

(52) U.S. Cl. 607/88

(57) ABSTRACT

A light therapy system provides for self-alignment or positioning with respect to a joint of a subject. The light therapy system can provide light therapy to a body part of a subject. The therapy system has a main body configured to be placed adjacent a target site and an activatable light emitting system is coupled to the main body. The light emitting system is capable of delivering a therapeutic amount of light energy to the target site when the main body is placed adjacent the target site.

### CROSS-REFERENCE TO RELATED APPLICATION

This application claims the benefit under 35 U.S.C. § 119(e) of U.S. Provisional Patent Application No. 60/728,556, filed Oct. 20, 2005, where this provisional application is incorporated herein by reference in its entirety.

### BACKGROUND OF THE INVENTION

1. Field of the Invention

This disclosure is generally related to medicinal or therapeutic treatment systems, and more particularly to external wearable light therapy treatment systems.

2. Description of the Related Art

Light therapy devices that are intended for home-use typically require the user to accurately position the device, particularly for joint treatments. Light therapy devices often have very localized light sources that require precise positioning, thus requiring placement by highly-trained personnel. These types of devices are not suitable for self-administered light therapy by the average user because it is often difficult to ensure proper compliance, and the average user is not properly trained to administer light therapy. Other types of light devices, such as “light blankets,” do not require accurate placement, but they do not provide sufficient energy to the target for an effective therapeutic treatment.

### BRIEF SUMMARY OF THE INVENTION

In one embodiment, this problem is resolved by providing a structure that accommodates a joint of the subject or end user as a self-locating feature, to correctly position an array of lights sources for providing light therapy, for example, for pain relief. The structure may be sized, shaped and/or dimensioned to receive the joint when the joint is partially or fully bent or articulated. Bending the joint (e.g., knee) slightly may also expand the joint and improve the ability of the light to reach the targeted areas. In addition, this allows the targeting of the synovium, a novel target for light therapy for joint pain.

In one embodiment, a light therapy device is coupled to a brace, which conformally receives a joint of the subject, and thereby provides a desired alignment between the light sources and a treatment area. The light therapy patch conforms to a non-planar portion of a subject's body at a treatment site to which the therapy is to be administered. The light therapy patch includes a flexible substrate formed of a dielectric material. Included within the flexible substrate are a plurality of open perforations that extend therethrough to provide ventilation paths enabling movement of air and moisture.

A power source is coupled to the patch for supplying an electrical current at a desired voltage to a plurality of flexible conductive traces that are applied to at least one surface of the flexible substrate. The flexible conductive traces define an electrical circuit for conveying an electrical current provided by the power source to portions of the flexible substrate. A plurality of light emitting sources are mounted to the flexible substrate in a spaced-apart array and are electrically coupled to the conductive traces to receive the electrical current. The electrical current energizes the plurality of light emitting sources so that they emit light to provide the light therapy at the treatment site.

The plurality of conductive traces may be produced by applying a conductive material, media, or fluid (e.g., a conductive ink) to the surface of the flexible substrate. If a conductive fluid is used, the conductive traces may be formed when the conductive fluid sets, becoming a flexible solid.

An adhesive may be provided to secure the flexible substrate to the non-planar portion of the subject's body and/or to the brace or other structure, so that the flexible substrate conforms to the non-planar portion. Adhesive may be applied to a surface of the flexible substrate opposed to the non-planar portion of the subject's body, to adhere the flexible substrate to the brace or other support. Adhesive may also be applied either to the non-planar portion of the subject's body before applying and conforming the flexible substrate to said non-planar portion, or is disposed on a surface of the flexible substrate that faces toward the non-planar portion of the subject's body when the flexible substrate is applied thereto.

Optionally, a light reflective layer disposed over an outwardly facing surface of the flexible substrate is provided to reflect light emitted by the light sources back toward the treatment site. Also, an optically transparent coating is preferably applied over the plurality of light sources mounted on the flexible substrate to provide protection.

The power source may comprise a flexible polymeric battery. A lead connects the flexible polymeric battery to the plurality of conductive traces, and the flexible polymeric battery is carried by the subject separate from the flexible substrate during administration of the light therapy.

The plurality of light emitting sources may emit a broad spectrum light. The plurality of light emitting sources may, for example, take the form of incandescent, halogen, fluorescent, electroluminescent sources, or some type of light emitting diodes, such as polymeric light emitting diodes, organic light emitting diodes, and/or metallic light emitting diodes.

The electrical circuit on the patch may comprise a plurality of parallel circuits conveying the electrical current to groups of the light sources, so that each group is separately energized by the electrical current. A microcontroller may be coupled to the electrical circuit for separately controlling the electrical current supplied to each group of light sources to control an intensity of the light administered to different regions of the treatment site.

Another embodiment is directed to a method of aligning and administering a light therapy to a particular treatment site.

In one embodiment, a device for providing light therapy includes a conformal flexible light emitting patch and a brace or wrap for positioning the conformal flexible light emitting patch with respect to a treatment site. The brace or wrap may, or may not include a hinge, and may or may not include fasteners, for example one or more straps, with or without hook and loop fasteners. The straps can form a mounting system.

In some embodiments, a light therapy treatment system comprises a power source and a wearable positioning structure. The positioning structure is configured to receive a body part and to engage at least one anatomical feature of the body part so as to position itself with respect to the body part. A light emitting system is coupled to the power source. The light emitting system is positioned relative to the positioning structure such that, when the body part is received by the positioning structure and the light emitting system receives energy from the power source, the light emitting system is positioned (e.g., due to engagement between the positioning structure and the at least one anatomical feature) to deliver a therapeutically effective amount of light to a target site in the body part.

In some other embodiments, a treatment system for providing light therapy to a joint of a subject comprises a joint brace including a main body configured to be placed adjacent the joint and an activatable light emitting system coupled to the main body. The light emitting system is capable of delivering a therapeutic amount of light energy to the joint when the main body is placed adjacent the joint.

In other embodiments, a treatment system for providing therapy to a treatment site of a subject is provided. The system comprises a wearable main body configured to be placed at least proximate the treatment site and an activatable light emitting system coupled to the main body. The light emitting system is capable of delivering a therapeutic amount of light energy to the treatment site. The system further includes an activatable non-light penetrating energy system coupled to the main body. The activatable non-light penetrating energy system is capable of delivering a therapeutic amount of non-light energy to the treatment site.

In some embodiments, a method of providing light therapy to at least one target site in a body part of a subject is provided. The method includes determining at least one anatomical feature of the body part based on a location of the at least one target site. The body part is placed in a positioning structure of a therapy treatment system. The positioning structure has a characteristic configuration. The light emitting system of the therapy treatment system is aligned with the at least one target site in the body part by engaging the positioning structure with the at least one anatomical feature of the body part. The light emitting system is operated to deliver a therapeutically effective amount of light to the at least one target site while the positioning structure maintains its characteristic shape to align the light emitting system with the target site.

In some other embodiments, a method of providing light therapy to a joint of a subject is provided. The method includes placing a joint having arthritis in proximity to a wearable therapy treatment system and delivering a dose of high intensity light to the joint. The dose comprises a therapeutically effective amount of high intensity light to substantially inhibit progression of at least one condition associated with arthritis. In some embodiments, the at least one condition can include, without limitation, discomfort, pain, inflammation, warmth, or cartilage damage or destruction. In some embodiments, the therapeutically effective amount of high intensity light substantially prevents or reverses the progression of at least one condition associated with arthritis.

In some other embodiments, a method of providing light therapy to a joint of a subject is provided. The method includes delivering a dose of high intensity light to the joint. In some embodiments, the dose comprises a therapeutically effective amount of high intensity light to substantially inhibit progression or to decrease an inflammatory response of at least one condition associated with an inflammation of the joint.

In some other embodiments, a method of providing light therapy to a joint of a subject is provided. The method includes placing a joint in proximity to a wearable therapy treatment system and delivering a dose of high intensity light to the joint. In some embodiments, the dose comprises a therapeutically effective amount of high intensity light to inhibit cartilage destruction. In some embodiments, the dose comprises a therapeutically effective amount of high intensity light to induce cell proliferation in cartilage.

In some embodiments, a light therapy device comprises a conformable light therapy patch and a structure. The light therapy patch includes a substrate sufficiently flexible to conform to a non-planar portion of a subject that is to receive light therapy. A plurality of light emitting sources is coupled to the substrate, and at least one circuit electrically couples at least some of the light sources. The structure is configured to support the conformable light therapy patch while accommodating a joint of a subject that is to receive light therapy.

In yet other embodiments, a method of providing light therapy to a non-planar area of a subject comprises placing a conformable light therapy patch in a support structure. The support structure is secured to the subject with the opposed to the non-planar area of the subject. A plurality of light emitting sources of the conformable patch is operated to deliver light therapy to the non-planar area of the subject.

Noninvasive techniques can treat target sites at different depths and positions in an individual's body. The target sites can include, without limitation, damaged tissue, inflamed tissue, diseased tissues (e.g., cancerous cells), interstitial tissues, epithelial tissues, connective tissues (e.g., blood, cartilage, and/or bone), nerve tissues, or other regions of interest. The target site can be treated with or without using medicaments or treatment agents. For example, the disclosed embodiments can treat joint tissues with or without utilizing photosensitive agents or other energy activated agents. Joint tissue can include, without limitation, bone, cartilage, synovium, capsule, tendon, muscle, ligament, and/or nerve.

Light therapy, however, can involve treatment agents, e.g., photosensitive agents, energy activated agents, and/or drugs and compounds to specific target cells or compositions of a subject or patient. Light or non-light energy (e.g., ultrasonic energy) at a relatively low intensity rate can be administered over a prolonged period of time in order to activate these agents. These sources may achieve maximal cytotoxicity with minimal side effects.

Various types of light therapy treatment systems can be used for diagnostic, therapeutic, cosmetic, or other types of procedures. In some diagnostic applications, the light therapy treatment systems can emit light with a wavelength selected to cause the photo-reactive agent to fluoresce as a means to acquire information about the targeted cells without damaging the targeted cells. In some therapeutic and cosmetic applications, the wavelength of the light delivered to the targeted cells treated with the photo-reactive agent causes the agent to undergo a photochemical reaction with oxygen in the localized targeted cells, to yield free radical species (such as singlet oxygen), which cause localized cell destruction (e.g., cell lysis), size reduction, or necrosis, for example.

A photoreactive or photosensitizing agent having a characteristic light absorption waveband can be administered to the patient, either orally or by injection or even by local delivery to the treatment site. The photoreactive or photosensitizing agent is subsequently selectively absorbed by abnormal tissue much more so than by normal tissue. Once the abnormal tissue has absorbed or linked with the photoreactive or photosensitizing agent, the abnormal tissue can then be destroyed by administering light of an appropriate wavelength or waveband corresponding to the absorption wavelength or waveband of the photoreactive agent.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is an elevational side view of an external light therapy treatment system worn by a subject, according to one illustrated embodiment.

FIG. 2 is an elevational side cross-sectional view of the light therapy treatment system of FIG. 1, according to one illustrated embodiment.

FIG. 3 is a pictorial view of a self-aligning light apparatus having a mounting system in an opened position.

FIG. 4 is a pictorial view of a self-aligning light apparatus having a mounting system in a closed position, according to one illustrated embodiment.

FIG. 5A is an elevational side cross-sectional view of a self-aligning light apparatus, according to one illustrated embodiment.

FIG. 5B is an elevational side cross-sectional view of a self-aligning light apparatus, according to one illustrated embodiment.

FIG. 5C is an elevational side cross-sectional view of a light therapy treatment system, according to one illustrated embodiment.

FIG. 6 is a plan view of a portion of a mounting system, according to one illustrated embodiment.

FIG. 7 is a cross-sectional view of a light emitting patch of the mounting system of FIG. 6 taken along line 7-7.

FIG. 8 is a cross-sectional view of a light emitting patch, according to one illustrated embodiment.

FIG. 9 is an elevational side view showing the use of a light emitting system positioned on a portion of a subject receiving therapy, according to one illustrated embodiment.

FIG. 10A is an elevational side view of a light therapy treatment system positioned on a knee, according to one illustrated embodiment.

FIG. 10B is an elevational side view of a hinged light therapy treatment system positioned on a knee, according to one illustrated embodiment.

FIG. 11 is an elevational front view of the light therapy treatment system of FIG. 10A.

FIG. 12 is a pictorial view of a pack of the light therapy treatment system of FIG. 10A.

FIG. 13 is a pictorial view of an external light therapy treatment system, according to one illustrated embodiment.

FIG. 14 is an elevational side view of an external light therapy treatment system worn on a wrist of a subject, according to one illustrated embodiment.

FIG. 15 is a cross-sectional side view of the light therapy treatment system of FIG. 14.

FIG. 16 is a pictorial view of an external light therapy treatment system worn on a wrist of a subject, according to one illustrated embodiment.

FIG. 17 is a pictorial view of treatment areas for light therapy, according to one illustrated embodiment.

FIG. 18 is an elevational side view of an external light therapy treatment system worn on a wrist of a subject, according to one illustrated embodiment.

FIG. 19 is a cross-sectional side view of the light therapy system of FIG. 18.

FIG. 20 is a pictorial view of an external light therapy treatment system worn on a wrist of a subject, according to one illustrated embodiment.

FIG. 21 is a cross-sectional view of the external light therapy treatment system of FIG. 20.

FIG. 22 shows an anatomical feature of a hand used to position an external light therapy treatment system, according to one illustrated embodiment.

FIG. 23 is an elevational side view of an external light therapy treatment system worn on an elbow of a subject, according to one illustrated embodiment.

FIG. 24 is an elevational cross-sectional view of the light therapy treatment system of FIG. 23.

FIG. 25 shows an anatomical feature of an elbow used to position an external light therapy treatment system, according to one illustrated embodiment.

FIG. 26 shows another anatomical feature of an elbow used to position an external light therapy treatment system, according to one illustrated embodiment.

FIG. 27 is a pictorial view of an external light therapy treatment system worn on a hand of a subject, according to one illustrated embodiment.

FIG. 28 is a plan view of an external light therapy treatment system for treating a foot, according to one illustrated embodiment.

FIG. 29 is an elevational side view of the light therapy treatment system of FIG. 28.

FIG. 30 is a pictorial view showing treatment areas of a foot.

FIG. 31 is an elevational side view of an external light therapy treatment system worn on an ankle of a subject, according to one illustrated embodiment.

FIG. 32 is a rear elevational view of the light therapy treatment system of FIG. 31.

FIG. 33 is a pictorial view of an external light therapy treatment worn on a head of a subject, according to one illustrated embodiment.

FIG. 34 is a pictorial view of an external light therapy treatment worn on a hip of a subject, according to one illustrated embodiment.

FIG. 35 is a pictorial view of an external light therapy treatment worn on a shoulder of a subject, according to one illustrated embodiment.

FIG. 36 is a pictorial view of an external light therapy treatment worn on a torso of a subject, according to one illustrated embodiment.

FIG. 37 is a cross-sectional side elevational view of a portion of a flexible patch for administering light therapy, according to one illustrated embodiment.

FIG. 38 is a schematic plan view of an undersurface of a portion of the flexible patch, according to one illustrated embodiment.

FIG. 39 is a schematic plan view of the outer surface of the flexible patch and a flexible power source used to provide electrical current to the flexible patch, according to one illustrated embodiment.

FIG. 40 is an enlarged view of the inner surface on a portion of the flexible patch, according to one illustrated embodiment.

FIG. 41 is a plan view of the inner surface of a flexible patch showing a central group of light sources and a peripheral group of light sources, according to one illustrated embodiment.

FIG. 42 is a schematic block diagram illustrating the functional components of a microcontroller for the flexible patch, according to one illustrated embodiment.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with light delivery devices, control circuits, power regulators and/or light emitting sources, for example incandescent light sources or light emitting diodes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims, the singular forms “a,” “an,” and “the” include plural referents unless the content clearly dictates otherwise. Thus, for example, reference to a light emitting patch including “a light source” includes a single light source, or two or more light sources. It should also be noted that the term “or” is generally employed in its sense including “and/or” unless the content clearly dictates otherwise.

As used herein and in the claims, the term “subject” generally refers to any host or animal, and includes, without limitation, mammals, such as horses, dogs, cats, and particularly humans.

The headings and Abstract of the Disclosure provided herein are for convenience only and do not interpret the scope or meaning of the embodiments.

The following description relates to treatment systems such as orthopedic appliances used to, for example, support, align, or hold a body part in a desired position. These treatment systems provide light therapy to the body part. Exemplary light therapy treatment systems include, without limitation, braces, supports, footwear, hand protectors, gloves, devices that support joints (e.g., normal and abnormal joints), devices that correct abnormal curves in the spine, devices that provide support to prevent injury or limit pressure on a joint to allow the joint to heal, and the like. For purposes of this description and for clarity, an external light therapy treatment system will be described and then a description of its components and methods of use will follow. Light therapy treatment systems are disclosed in the context of providing light therapy to movable joints because they have particular utility in this context. However, the light delivery systems can be used in other contexts to treat other regions of the body.

Light Therapy Treatment System

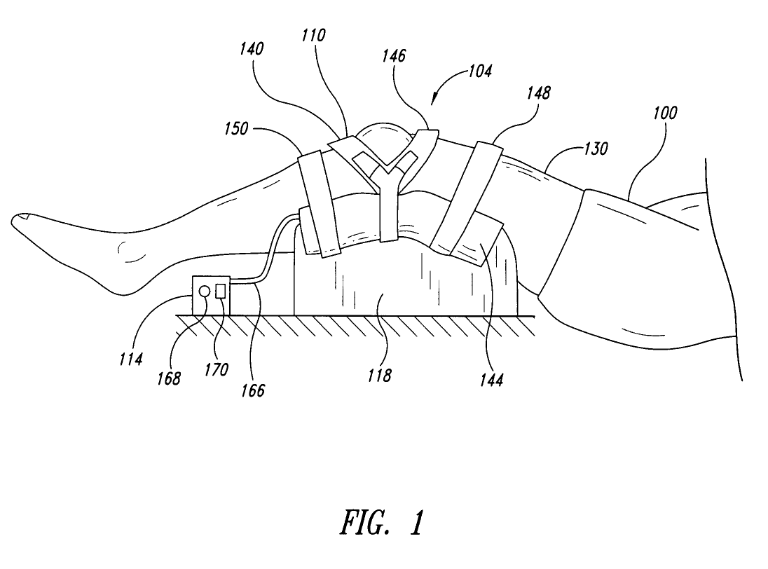

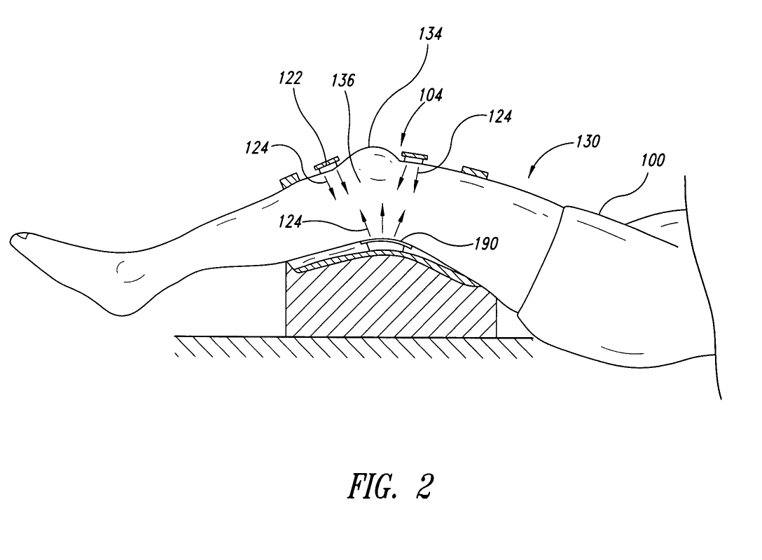

FIG. 1 shows a subject 100 wearing a light therapy treatment system 104 for providing light therapy to one or more target sites. The illustrated light therapy treatment system 104 includes a self-aligning light apparatus 110, a controller 114 coupled to the self-aligning light apparatus 110, and a support member 118 positioned beneath the light apparatus 110. To perform light therapy as shown in FIG. 2, a light emitting system 122 of the light apparatus 110 can deliver light (indicated by the arrows 124) to one or more target sites in the subject 100. Light therapy can be performed on the illustrated body part 130, for example in the form of a leg, to treat injuries and damaged tissue, promote tissue regeneration, reduce inflammation, and the like.

To enhance access to internal tissue, for example, within a knee 134, the treatment system 104 is configured to maintain the knee 134 at a predetermined position, or within a predetermined range of positions, to improve the efficacy of the therapy session. The position of the knee 134 can be adjusted to perform different procedures on tissues at different locations. For example, the treatment system 104 can position the knee 134 at a first configuration to perform focused light therapy on cartilage. Another light therapy treatment system can be used to position the knee 134 at a second configuration different than the first configuration to perform focused light therapy on synovial tissue (e.g., the synovial membrane and/or synovial fluid) in the knee 134.

The illustrated treatment system 104 of FIGS. 1 and 2 locates itself using anatomical features, shape, geometry, and/or dimensions of the leg 130 to deliver light 124 to position and orient the light therapy treatment system 104, thereby reducing the level of skill required to properly perform light therapy. The illustrated treatment system 104 is therefore well suited for use by typical medical personnel, users at home (e.g., to perform self-administered therapy), and other average users.

Traditional light delivery devices, such as light pads, rely on proper placement by a well-trained user. Additionally, these types of light delivery devices also have a tendency to migrate relative to treatment sites, especially when used for extended periods of time. The treatment system 104, however, conformally accommodates the bent knee 134, with the conformable flexible light emitting system 122 positioned on the front and rear of the knee. The size, shape and dimensions of the treatment system 104 assures that the conformable flexible light emitting system 122 is correctly positioned with respect to the treatment site. The size, shape and dimensions of the treatment system 104 may also ensure that the knee joint 136 is properly bent, neither under, nor over extended.

The self-aligning light apparatus 110 of FIG. 1 can use one or more natural anatomical features, for example, of the leg 130, to assist in placement, alignment and/or ongoing positioning of the apparatus 110. The concept applies to the knee, wrist, elbow, ankle, hip, shoulder, finger, and any other joint or body part that has an identifiable feature (e.g., a bend point, flexion crease, unique topological features, and the like) that can be referenced to position the system 104 and target the therapy.

With continued reference to FIG. 1, the self-aligning light apparatus 110 includes a positioning structure 144 for receiving and accommodating the leg 130 and a mounting system 140 for securing the positioning structure 144 to the subject 100. The illustrated light apparatus 110 can surround and circumscribe the periphery of the leg 130, thereby accurately and effectively positioning the light emitting system 122 with respect to treatment sites. The light emitting system 122 can then be used to delivery a therapeutic amount of light energy to treatment sites, for example target sites beneath or adjacent to the highly contoured dermis of the leg 130.

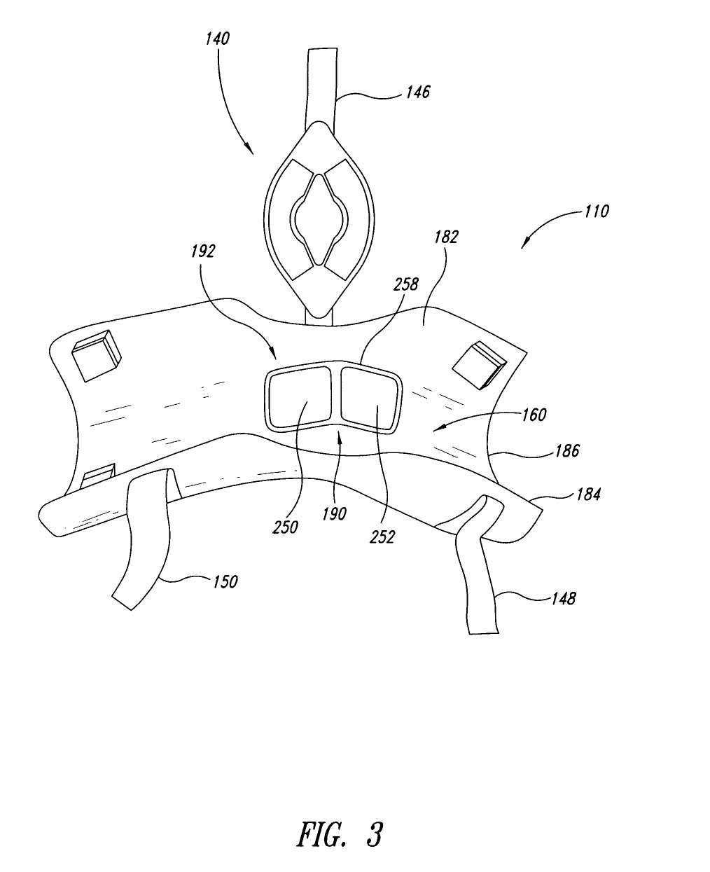

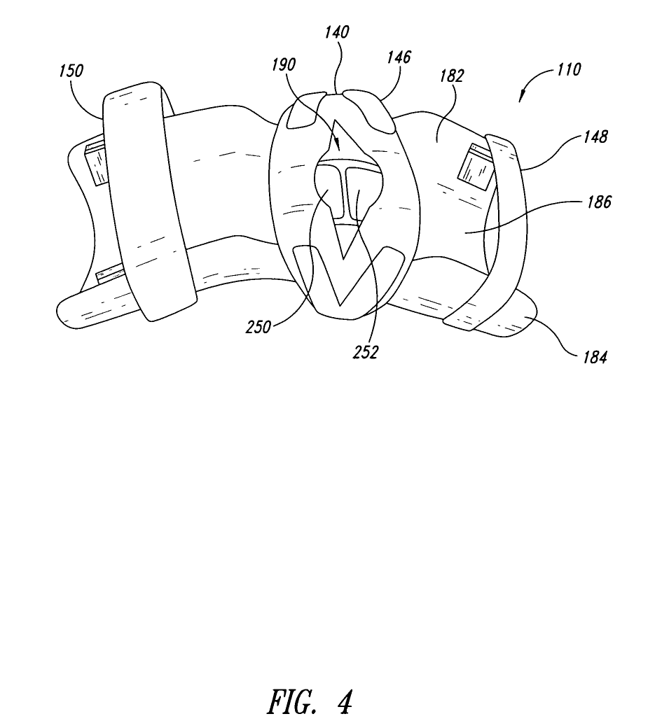

The mounting system 140 is movable between an open position (FIG. 3) and a closed position (FIGS. 1, 2, and 4). The illustrated mounting system 140 includes a knee strap system 146, a thigh strap 148, and a leg or gastrocnemis strap 150. When the mounting system 140 is in the closed position, at least a portion of the light emitting system 122 can be in direct contact with the leg 130 for efficiently transmitting light energy to and through at least a portion of the leg 130.

Referring to FIG. 3, when the mounting system 140 is in the open position, the subject's leg 130 can be conveniently inserted into a receiving portion 160 of the positioning structure 144 such that the positioning structure 144 surrounds a posterior portion of the leg 130, including the knee 134. The mounting system 140 is then brought around the anterior portion of the leg 130 to the closed position to hold the positioning structure 144 snuggly against the leg 130.

The light therapy treatment system 104 can securely hold the leg 130 to reduce, limit, or substantially eliminate unwanted movement between the light emitting system 122 and the target sites, thereby minimizing misalignment. In some uses, it is important to keep the light emitting system 122 somewhat fixed relative to a target site for a threshold length of time for an effective treatment session. The mounting system 140 and positioning structure 144 can cooperate to generally fix the light emitting system 122 relative to the target tissue, thus providing an effective treatment session. Additionally, the light emitting system 122 can be pressed against the leg 130 to ensure efficient delivery of light to the treatment sites, as note above.

In the illustrated embodiment of FIG. 1, the knee strap system 146, thigh strap 148, and leg strap 150 extend across the front of the knee joint, thigh, and leg, respectively. The mounting system 140 can be adjustable to accommodate legs varying in size and geometry. The lengths of the strap system 146 and straps 148, 150, for example, can be increased or decreased to decrease or increase, respectively, the pressure applied to the leg 130. Any number of straps, belts, harness systems, or other retaining devices can be used alone or in combination to hold the self-aligning light apparatus 110 in a desired position.

The illustrated self-aligning light apparatus 110 in FIGS. 1 and 2 rests upon the support member 118 to elevate the leg 130. The support member 118 can be sufficiently compliant to deform and accommodate the shape and contours of the light apparatus 110 and leg 130. In some embodiments, the support member 118 is a cushion or pad that has a preset shape suitable for receiving the light apparatus 110 when the subject 100 is lying down, for example, in bed or on a sofa. Other types of support members can also be used.

The self-aligning light apparatus 110 can also be used without the support member 118. For example, the light apparatus 110 alone may be used on the subject 100 in a sitting position. Thus, light therapy can be conveniently performed on a subject when the subject is at work, traveling, watching television, or performing other everyday activities.

The light therapy treatment system 104 of FIG. 1 can be used to perform various types of light therapies. Light therapy is broadly construed, in some embodiments, to include delivering light to stimulate, activate, or otherwise excite one or more areas of targeted tissue. In some embodiments, a therapeutic amount of light is used to effect (e.g., photo-activate or photo-excite) one or more target sites by subjecting the one or more target sites to one or more wavelengths of light that are approximately close to, if not equivalent to, at least one excitation wavelength of the cells at the target site. It is understood that even if one site is “targeted,” it is possible that other cells in a vicinity of the targeted cell may also be subjected to light and likewise treated. In some embodiments, the light therapy procedure can affect an insubstantial percentage of non-targeted cells in the vicinity of the targeted cells. Advantageously, light therapy may not adversely affect healthy non-targeted cells even though light is transmitted through the healthy non-targeted cells.

Light therapy can be used to treat various types of conditions, diseases, symptoms, and/or problems to, for example, reduce or alleviate pain for pain management, slow or limit the progression of the condition or disease, promote healing, minimize unwanted symptoms, and the like. In some embodiments, light therapy may be used to cure a disease without any appreciable adverse side effects.

Generally, by using noninvasive light therapy techniques, the light therapy treatment systems disclosed herein can treat target sites at different depths and positions in the subject's body. The target sites can include, without limitation, diseased tissues (e.g., inflamed tissue), interstitial tissues, epithelial tissues, connective tissues (e.g., tendons, ligaments, cartilage, and/or bone), body fluids (e.g., blood), tissue attachments, muscles, nerve tissues, or other regions of interest. A target site can be treated with or without using medicaments or treatment agents.

Treatment parameters for the system 104 can be selected based on the diagnosis of the subject, and may include, without limitation, power density, treatment type, treatment duration or period, depth of penetration, pulse intensity, pulse duration, pulse repetition rate, stop-start time, position and orientation of the light emitting system 122, and the like. Additional treatment parameters can also be used.

The controller 114 can be used to set the treatment parameters during the light therapy session. The treatment parameters can be based, at least in part, on dimensions or measurements (e.g., joint size, range of motion, optical measurement of arthritis markers, internal fluid pressures, tissue properties, tissue cartilage hardness, and the like) of the subject 100. Physical measurements can be used to determine an appropriate light therapy routine. Additionally, user feedback (e.g., level of pain or discomfort) can be used to optimize and develop an interactive therapy plan. The appropriate light dosages can also be determined using clinical variables, such as measurements of skin color, distance through tissue to target sites, composition of the tissue (e.g., fat, muscle, bone, etc.), degree of pain, and the like. Time variables can be used in course therapy treatments that require, for example, maintenance of dose.

In some embodiments, the power density in the target tissue can be maintained at or above a threshold level known to have beneficial responses at the target site. Threshold levels can vary for different types of tissues to account for properties (e.g., optical properties) of the tissues. The position of the treatment system 104 can be selected to achieve the power densities in the tissues based on their wavelength absorption properties or other physical characteristics.

Tissue edema (often associated with soft tissue strain, soft tissue stress, arthritis, blunt trauma, or surgical procedures) can be treated with light therapy. In some embodiments, the treatment system 104 can treat joints suffering from arthritis, osteoarthritis, rheumatoid arthritis, juvenile rheumatoid arthritis ankylosing spondylitis, psoriatic arthritis, mixed connective tissue disease, combinations thereof, and other related conditions or diseases leading to degeneration of a joint or loss of cartilage. Mixed connective tissue diseases include, without limitation, immune-related connective tissue diseases including systemic lupus erythematosus, rheumatoid arthritis (noted above), scleroderma, and polymyositis. Light therapy can be used to promote cell regeneration or proliferation (e.g., cartilage proliferation), reduce inflammation of the joint, treat energy depleted tissue, and the like. Energy depleted tissue can result from tissue hypoxia, contusions (e.g., subcutaneous contusions), and the like.

Injured or unhealthy soft tissue and muscles can be a source of pain and discomfort. Light therapy can advantageously treat pain and discomfort often associated with, for example, tendonitis, carpal tunnel syndrome, epicondylitis (e.g., medial epicondylitis or lateral epicondylitis), tennis elbow, damaged rotator cuff, golfer's elbow, and other related conditions associated with tendons or other connective tissue. Light therapy can be performed either while the subject is active (e.g., playing sports, working, or performing activities that are closely associated with condition or disease) or while the subject is inactive (e.g., sleeping or resting).

In some embodiments, light therapy can be directed to spot inflammation (or a tender spot) often found with carpal tunnel syndrome, trigger point, or other similar conditions. The subject may actively aim the light energy towards the area or region of pain or discomfort. Even if an area or region of pain or discomfort has not been examined by a physician, the subject may still provide therapy that may reduce, limit, or substantially eliminate the pain or discomfort. Because light therapy does not adversely affect healthy tissue, users can employ light therapy to treat conditions, diseases, or symptoms that may not have been identified with a desired degree of certainty.

Light therapy can also be used to treat bones suffering from osteogenesis imperfecta, osteonecrosis, and other bone conditions or diseases reducing bone density, bone strength, and the like. Other known conditions, diseases, or problems can also be treated by using light therapy. The configuration of the light therapy treatment system can be selected based on the body part and type of light therapy to be performed.

With reference again to FIG. 1, the light therapy treatment system 104 can be programmed to perform various-types of light therapy. For example, one or more treatment parameters can be inputted into the controller 114, which in turn determines an appropriate light therapy program for the therapy session.

The controller 114 of FIG. 1 is coupled to the self-aligning light apparatus 110 via a wire connection or wireless connection 166. The wireless connection 166 can be configured to provide optical communication, radio frequency communication, ultraviolet communication, BLUETOOTH® communication, and other types of communications.

The controller 114 can accurately control the output of the light emitting system 122 to achieve the desired light energy treatment. As used herein, the term “controller” is a broad term and includes, without limitation, a device or system that can command an electrically powered device. Controllers may include, without limitation, one or more processors, microprocessors, digital signal processors (DSP), application-specific integrated circuits (ASIC), microcontroller circuit (see FIG. 42), and the like. To store information (e.g., light therapy programs, patient data, and the like), controllers may also include one or more storage devices, such as memory, read-only memory (ROM), random access memory (RAM), and the like.

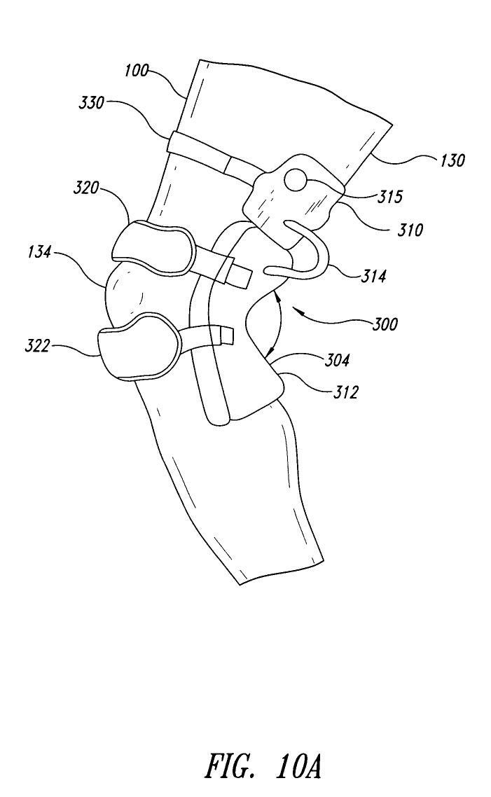

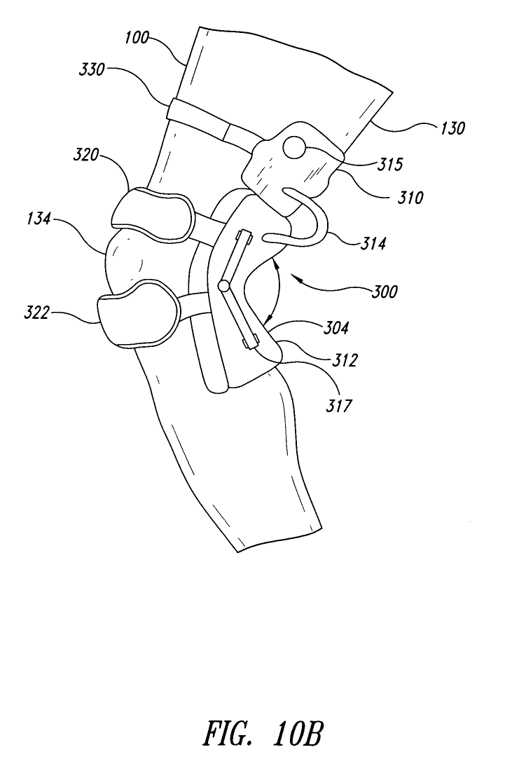

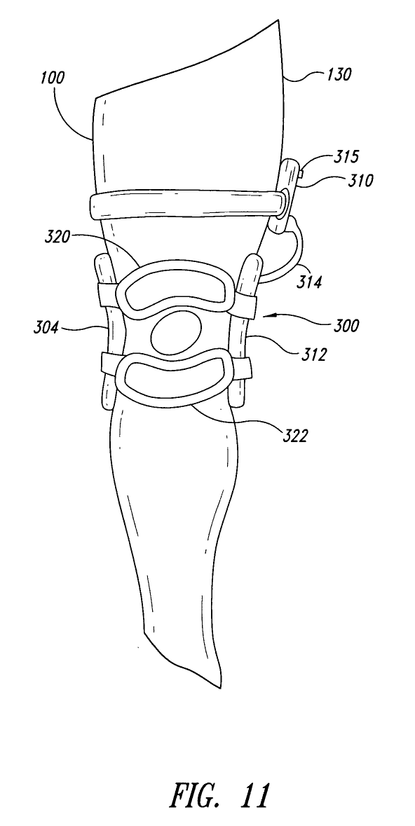

The controller 114 of FIG. 1 may further include one or more input devices 168 (e.g., an input display, keyboard, touchpad, controller module, or any peripheral device for user input) and one or more displays 170. The illustrated controller 114 also contains an internal power supply (e.g., one or more batteries or other type of power storage device) for powering the light emitting system 122 or other component of the system 104. In other embodiments, the light emitting system 122 can be powered by an AC power source, such as an AC outlet. Additional exemplary power sources include, without limitation, at least one of the following power sources: wall outlet, battery, computer ports (e.g. USB-type ports), DC vehicle/car outlet, solar (e.g., a solar panel), and portable electronic devices, as well as other power sources disclosed herein. Additional means for powering one or more components of the treatment system 104 are discussed in connection with FIGS. 10A to 12.

The positioning structure 144 of FIG. 1 can help maintain proper positioning and orientation of the leg 130 during light therapy. For some types of light therapy, the positioning structure 144 can minimize, limit, or substantially prevent unwanted movement of the leg 130, thereby maintaining the knee joint 136 at or near a preset flexion angle. For example, the positioning structure 144 can be a generally rigid member that biases, urges, or otherwise moves the knee joint 136 to a desired preset flexion angle. In other embodiments, the knee joint 136 is allowed to move within a range of flexion angles. The subject 100 may be able to apply forces sufficient to move the knee joint 136 outside of the preset range of knee flexion angles, if needed or desired. The positioning structure 144, however, can bias the knee 134 back towards the desired position, or range of positions. Unlike traditional light pads that merely surround a body part, the light therapy treatment system 104 attempts to position the body part so as to facilitate properly light delivery and, consequently, provide a more effective treatment.

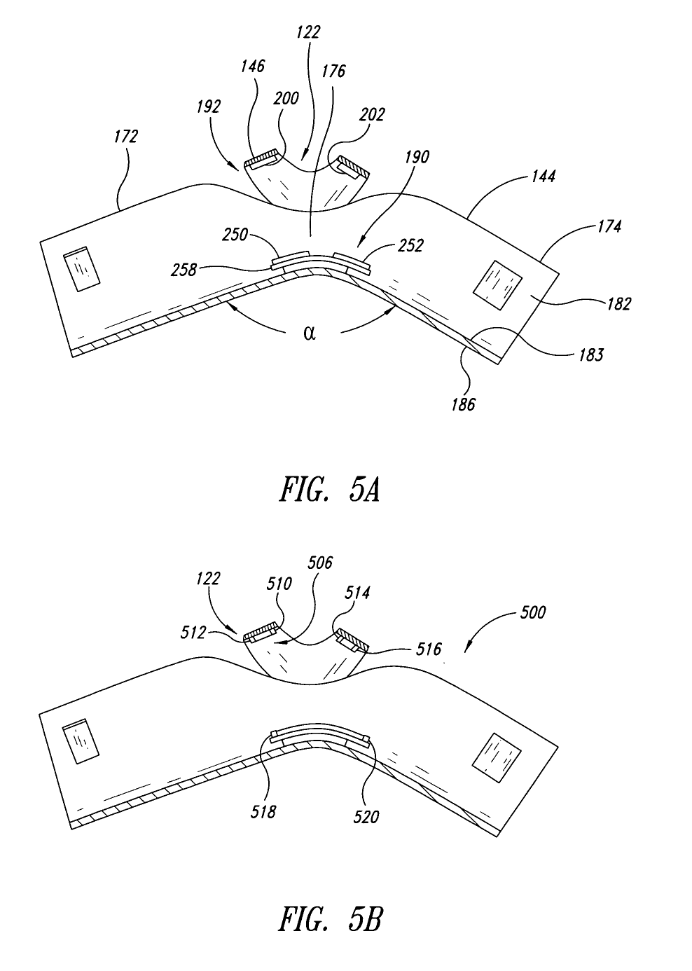

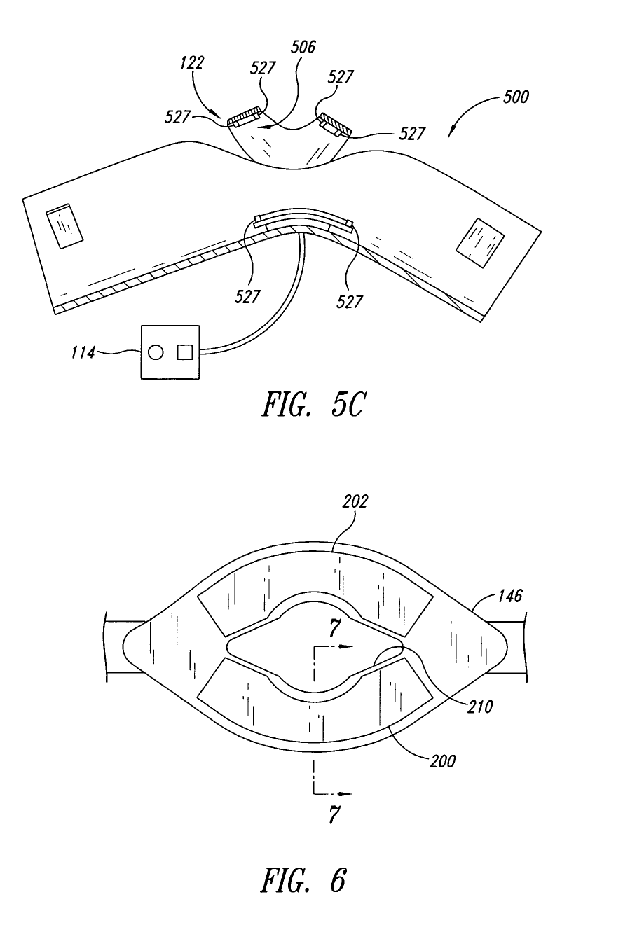

The illustrated positioning structure 144 of FIG. 5A includes a lower elongate portion 172, an upper elongate portion 174 and an angled or curved portion 176 therebetween. The lower elongate portion 172 and upper elongate portion 174 are configured to receive and hold the lower leg and thigh, respectively. The angled portion 176 receives the posterior portion of the knee 134. The configuration of the angled portion 176 determines the angle α defined by the lower and upper elongate portions 172, 174. The flexion angle of the knee joint 136 can be approximately equal to or similar to the angle α. As shown in FIG. 2, for example, the configuration of the leg 130 generally matches the configuration of the positioning structure 144.

The angle α can be selected based on the light therapy to be performed. The angle α can be in the range of about 5 degrees to about 90 degrees. The angle of the knee joint 136 can be between various angles in this range to target different regions of synovial tissue (e.g., the synovial membrane and/or synovial fluid). Such embodiments permit effective delivery of light to deep internal tissues, even tissues surrounding the femur, tibia, fibula, and/or the patella. In some non-limiting embodiments, the angle α is in the range of about 5 degrees to about 30 degrees to preventing locking of the knee 134 in a rigid, straight-leg position. A sufficient amount of blood flow can be maintained in the leg 130 for a long light therapy sessions. In other embodiments, the angle α is in the range of about 10 degrees to about 50 degrees. These embodiments are especially well suited for delivering light to both the periphery of the meniscus and at least one bursa while maintaining the knee 134 at a comfortable position. The subject 100 can use the light therapy treatment system 104 when sitting in a chair, for example. In other embodiments, the angle α is in the range of about 40 degrees to about 70 degrees to advantageously deliver a relatively large amount of light to the inner portion of the meniscus and cartilage and ligaments. Where the meniscus is worn or otherwise damaged, focused light can be delivered to the worn or damaged portions to facilitate tissue regeneration or cell proliferation. In other embodiments, for example, the angle α is in the range of about 70 degrees to about 90 degrees to advantageously deliver light energy to a significant portion of the synovial fluid and/or synovial membrane and cartilage and ligaments. An appropriate treatment position of the leg 130 can be selected based on the tissue to be treated.

Other angles α are also possible to obtain the desired optical access to internal tissues of interest. In some embodiments, the angle α is equal to or greater than about 5 degrees, about 10 degrees, about 15 degrees, about 20 degrees, about 25 degrees, about 50 degrees, about 60 degrees, about 70 degrees, about 80 degrees, or about 90 degrees. The rate of energy delivery can be adjusted based on the position of the knee 134 and the position of the targeted tissue in the knee 134. In some embodiments, the knee is at full knee flexion during light therapy.

Referring to FIGS. 3 to 5, the positioning structure 144 has a generally semi-circular axial cross-sectional profile. The positioning structure 144 includes a pair of sidewalls 182, 184 and a curved base member 186 extending between lower ends of the sidewalls 182, 184. An upper surface 183 (FIG. 5A) of the positioning structure 144 provides a smooth curved surface for engaging the leg 130. The sidewalls 182, 184 can help guide the leg 130 into physical contact with the light emitting system 122.

To provide the desired level of joint fixation, the positioning structure 144 can be configured to closely receive the leg 130. For example, the pair of sidewalls 182, 184 are spaced sufficiently apart to receive the leg 130 of the subject 100, and when the mounting system 140 is in the closed position, the two sidewalls 182, 184 are sufficiently proximate to secure the positioning structure 144 to the leg 130.

One or more metals (e.g., aluminum, steel, stainless steel, titanium, and the like), plastics, polymers, composites, combinations thereof, or other similar materials can be used to form the illustrated positioning structure 144. In some embodiments, the positioning structure 144 is made of polypropylene, nylon, polyethylene terephthalate (PET), polyurethane, combinations thereof, and other polymers suitable for contact the subject's skin. One of ordinary skill in the art can select the materials to form a semi-rigid or rigid positioning structure 144.

In alternative embodiments, the positioning structure 144 can be in the form or one or more rods (e.g., flexible, semi-rigid, or rigid rods), stiffeners, or other elongated members that can position the leg 130 as desired. Mounting systems can couple such positioning structures to the leg 130.

The light emitting system 122 may have a posterior light system 190 and an anterior light system 192 for delivering light to and through a posterior portion and anterior portion, respectively, of the leg 130. The anterior light system 192 includes a lower light emitting patch 200 and an upper light emitting patch 202, each coupled to a face of the knee strap system 146 facing the positioning structure 144. As shown in FIG. 6, an opening 210 for allowing at least a portion of the kneecap (or patella) to pass therethrough is between the lower and upper light emitting patches 200, 202 such that the light emitting patches 200, 202 can generally surround the kneecap (see FIG. 2). The kneecap can protrude outwardly from the opening 210 to further help locate and position the light emitting system 122 relative to the leg 130.

The illustrated elongate strip-like lower and upper light emitting patches 200, 202 are adapted to conform closely to contours of the skin, even non-planar regions of skin. The term “patch” is broadly construed to include, without limitation, a somewhat flat device or system capable of covering an area of skin, whereby that device or system, when energized, can deliver a selected amount of light (e.g., a therapeutically effective amount of light) to at least one target site. In some embodiments, the patch can be twisted, bent, folded, or otherwise manipulated into a desired configuration. Such flexible, conformable patches can accommodate various portions of the subject's body to perform phototherapy on different target sites. A highly flexible patch can conform closely to highly contoured portions of the subject's body and, consequently, can provide efficient light transmission to the body. The patches disclosed herein can be replaced or combined with other types of energy sources, arrays of light sources, lasers, single light sources, and the like, but these alternative light sources may not provide an effective light distribution as compared to light emitting patches. In some embodiments, patches can be semi-rigid or rigid. A rigid patch can have a preset shape for providing support to a body part.

The patches 200, 202 can be generally similar to each other and, accordingly, the following description of one of the patches applies equally to the other, unless indicated otherwise. The patches 200, 202, in some embodiments, can be generally similar to or the same as the patches discussed in connection with embodiments described below.

With respect to FIGS. 2 and 5, the posterior light system 190 is configured for placement at or near the rearward portion of the knee 134. At least a portion of the posterior light system 190 can be configured to engage the popliteal fossa, i.e., the shallow depression at the back of the knee joint 136. Interaction between the posterior light system 190 and the popliteal fossa can help maintain alignment of the light emitting system 144 with the treatment site. In some embodiments, the posterior light system 190 can fit conveniently in the flexion crease of the knee joint. When placing the light therapy treatment system 104 on the leg 130, the posterior light system 190 can be mated with the popliteal fossa or flexion crease to ensure placement.

The leg 130 shown in FIG. 2 rests comfortably on the posterior light system 190. In other embodiments, however, the leg 130 can be spaced from the posterior light system 190 to reduce pressure applied to the back of the knee 134. In other embodiments, the posterior light system 190 can be removed so that the leg 130 can rest comfortable on the upper surface 183 of the positioning structure 144.

Referring now to FIGS. 3 to 5, the posterior light system 190 includes a pair of light emitting patches 250, 252 and a base member 258 (FIG. 5A) coupling the pair of light emitting patches 250, 252 to the positioning structure 144. The pair of patches 250, 252 are somewhat angled with respect to one another. In some embodiments, the patches 250, 252 define an angle that is approximately equal to the angle α of FIG. 5A.

In some alternative embodiments, the posterior light system 190 can have a single continuous light emitting patch that extends continuously along the length of the base member 258. The number, sizes, shapes, and positions of the light emitting patches can be selected based on the number, sizes, shapes, and positions of the target sites.

The illustrated base member 258 fixedly couples the pair of patches 250, 252 to the upper surface 183 of the positioning structure 144. The base member 258 can be a substantially rigid mounting structure that limits or substantially prevents movement of the patches 250, 252 relative to the positioning structure 144. In some other embodiments, the base member 258 is a flexible mounting structure that provides a relatively large amount of movement of the patches 250, 252 relative to the positioning structure 144. Other types of coupling arrangements can also be used. The patches 250, 252, for example, can be directly coupled to the positioning structure 144 with fasteners (e.g., screws, nut and bolt assemblies, and the like), rivets, staples, adhesives, bonding agents, or other suitable coupling means.

In some embodiments, the light emitting system 122 may not include the posterior light system 190. The anterior light system 192 alone may be able to effectively treat the leg 130. Alternatively, the light emitting system 122 may not include the anterior light system 192. For example, the light emitting system 122 may include only the posterior system 190 for highly localized therapy on the posterior region of the leg 130.

The patches disclosed herein can include an array of light sources, such as an array of diodes. Edge emitting LEDs, surface emitting LEDs, super luminescent LEDs, laser diodes, or other suitable light energy sources can be used. Patches or other light sources disclosed herein can emit appropriate wavelength(s) or waveband(s) suitable for treating the patient, with or without using a treatment agent, as noted above. If a treatment agent (e.g., a photo-reactive or photosensitive agent) is utilized, the light therapy is performed with radiation wavelength(s) or waveband(s) that correspond with, or at least overlap with, the wavelength(s) or waveband(s) that excite or otherwise activate the target tissue. The light therapy can be performed with or without photosensitive agents. If photosensitive agents are administered in conjunction with the light therapy, the photosensitive agents can often have one or more absorption wavelengths or wavebands that excite them to produce substances which damage, destroy, or otherwise treat target tissues of the patient.

For example, the patch 200 of FIG. 6 can be configured to emit light having one or more wavelengths in the red spectrum, near infrared spectrum, and/or infrared spectrum. The patches can emit light, for example, having a wavelength or waveband in the range from about 200 nanometers to 1,000 nanometers. In some embodiments, the light sources emit a wavelength or waveband in the range from about 300 nanometers to about 800 nanometers. In some embodiments, the patches emit a wavelength or waveband in the range from about 600 nanometers to about 700 nanometers. In one embodiment, for example, the patches emit radiation with a peak wavelength of 664 nanometers plus or minus 5 nanometers, even if a photoreactive agent is used. In other embodiments, for example, at least one of the patches emits radiation with a peak wavelength in the range of about 610 to about 650 nanometers for light therapy performed without utilizing a photoreactive agent. In some embodiments, the light can include red light (e.g., a wavelength of about 620 nanometers to about 670 nanometers) and near infrared (e.g., a wavelength of about 820 nanometers to about 904 nanometers). Other wavelengths combinations are also possible.

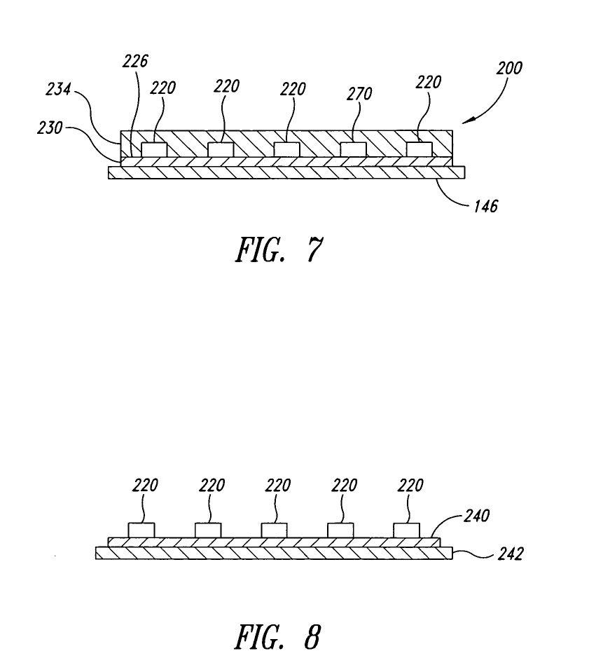

FIG. 7 shows the patch 200 having an array of light sources 220 configured to emit the same or similar wavelength or waveband. However, light sources 220 having different wavelengths or wavebands can be used to provide varying outputs. These light sources 220 can be activated simultaneously or at different times depending on the desired treatment. The light sources 220, for example, can also be activated and deactivated in a pulsed or timed sequence. Alternately, the control system 114 may be programmed to selectively activate and deactivate different sections of the array of the light sources 220. In this manner, a treatment protocol may be programmed into the control system 114. The treatment protocol can cause the light sources to be lit in a certain sequence and at a particular power level for a selected period of time.

The light emitting patches disclosed herein can have any number of light sources. In some embodiments, including the illustrated embodiment of FIG. 7, the patch 200 has five rows of light sources 220 generally evenly spaced from one another. Each row has light sources incrementally spaced along the length of the patch 220; however, a higher or lower number of light sources can be utilized based on the desired energy output, emitted wavelength(s) and/or waveband(s), dimensions of the target site, location of target site, desired level of energy penetration, and/or other treatment parameters.

In some embodiments, the light emitting patch 200 can output energy at energy levels equal to or greater than about 1 J/cm², 5 J/cm², 10 J/cm², 20 J/cm², 30 J/cm², 40 J/cm², 50 J/cm², or ranges encompassing such energy levels. The intensity of the outputted energy can be equal to or greater than about 5 mW/cm², 20 mW/cm², 50 mW/cm², or ranges encompassing such intensities. The intensity of the outputted energy can be equal to or greater than about 50 mW/cm², 100 mW/cm², 125 mW/cm², 150 mW/cm² or ranges encompassing such intensities. In yet other embodiments, the light emitting patch 200 outputs a high power density equal to or greater than about 160 mW/cm², 180 mW/cm², or 200 mW/cm². The number and position of the light sources 220 can be selected based on, for example, the desired energy output levels and light distribution.

With continued reference to FIG. 7, the light sources 220 are mounted upon an upper face 226 of the flexible sheet 230. Any suitable mounting means can be employed to temporarily or permanently couple the light sources 220 to the sheet 230. For example, adhesives, bonding material, fasteners, solder, or other coupling means can securely couple the light sources 220 to the sheet 230.

An optional protective layer 234 can encapsulate the light sources 220, and can be optically transparent in order to transmit light generated by the light sources 220 to the protective layer 234 which, in turn, transmits the light to the subject. Various types of optically transparent materials can form the protective layer 234. Where the patch 200 is applied to a highly contoured region of a body part, the protective layer 234 can comprise a flexible material such that the patch 200 can conform closely to the highly contoured region, as noted above. The material(s) forming the patch 220 can be selected to achieve the desired structural properties, thermal properties, electrical properties, optical properties, wear characteristics, and durability.

Alternatively, the light sources 220 of FIG. 8 are mounted on an upper surface 240 of the sheet 242. Advantageously, light from the sources 220 is delivered directly to the skin without passing through any intermediate structure. For example, the light sources 220 can be pressed directly against the subject for efficient light transmission, thereby improving the efficiency of the light therapy treatment.

Various types of wire bonding techniques can interconnect the light sources 220 of FIGS. 7 and 8. Wires can electrically couple the light sources 220 together. The light sources 220 can also be mounted in a flip chip arrangement. A flip chip is one type of integrated circuit (IC) chip mounting arrangement that does not require wire bonding between chips. Thus, wires or leads that typically connect a chip/substrate having connective elements can be eliminated to further reduce the profile of the distal tip. Generally, solder beads or other elements can be positioned or deposited on chip pads such that when the chip is mounted upside-down in/on a sheet (e.g., the sheet 230 or 242), electrical connections are established between conductive traces of the sheet and the chip. Other types of mounting arrangements and electrical connections are described in more detail below.

In some methods of using the treatment system 104, the system 104 can be configured to self-align to improve the accuracy of light delivery. Generally, the light therapy treatment system 104 can interact with the body part 130 to align the light emitting system 122 with the target site. In some embodiments, at least one anatomical feature or locator of the body part 130 is identified based on the location of the target site in the subject. Once a target site is determined, a corresponding anatomical feature can be identified and used for locating the light therapy treatment system 104. The body part 130 of the subject 100 is then placed in the positioning structure 144. The light emitting system 122 is aligned with the target site in the body part 130 by engaging the positioning structure 144 with the at least one anatomical feature. The emitting system 122 is then operated to deliver a therapeutically effective amount of light to the target site.

A variety of anatomical features of the leg 130 can be used to assist in the placement of the treatment system 104. Anatomical features can include, without limitation, joint motion, concave or convex surfaces, depressions, bend points, protruding features (e.g., protruding bones such as patella or fibula), physical relationships between body parts (e.g., the lower leg and thigh), and the like. Tapered or narrowed regions of the body are anatomical features that are especially well suited for receiving a mounting system 140. Other types of irregular surfaces or features on the subject can function as locators.

The joint motion and mechanical features of the light therapy treatment system 104 can be linked to assist in the placement and orientation of the light emitting system 122. The mechanical features can be anatomical feature locators, such as a conformable light patches or other structures, suitable for engaging the subject 100. Various types of mechanical features of the treatment system 104 can physically contact the subject 100 to facilitate proper positioning.

Once worn, the treatment system 104 is configured to maintain proper positioning with respect to the leg 130 before, during, and/or after the phototherapy procedure. The treatment system 104 can interact with one or more anatomical features to limit or substantially prevent unwanted migration of the light emitting system 122 relative to the target sites, thereby ensuring properly light delivery to the target sites. As noted above, the light therapy treatment system 104 can function as fixation device for generally fixating the knee joint 136. For example, the illustrated light therapy treatment system 104 of FIG. 1 can be in the form of a rigid knee brace that generally maintains permanent fixation of the knee joint 136 during light therapy.

It is anticipated that the treatment system 104 would be worn for greater than about 10 minutes. In some embodiments, the light therapy can be last about 10 to about 30 minutes. The therapy can be repeated once or twice a day, or 2-3 three times per week. In some treatment programs, therapy can be delivered for a treatment period in the range of about 15 minutes to about 30 minutes. This therapy can be performed once a day for about 2 weeks to about 3 weeks. If needed, the therapy can be performed multiple times a day (e.g., twice a day). The method of applying energy over an extended duration, rather than in discrete daily treatment sessions, may offer benefits. Typically, light therapy is delivered in a single dose which is repeated daily or 2-3× per week.

In some embodiments, the treatment system can deliver light at a high power density in the range of about 10 mW/cm² to about 200 mW/cm². Relatively large areas of tissue can be covered as compared to the prior art. For example, the treatment systems may be able to provide an area of coverage in the range of about 30 cm² to about 100 cm². Where the treatment system is used to treat a joint, a large portion or the entire synovium can be illuminated for a rapid and effective treatment.

The total energy delivered to the target site can be relatively high. In some embodiments, for example, the total energy delivered to the target site can be equal to or greater than about 200 J. In some embodiments, the total energy delivered to the target site is in the range of about 200 J to about 3000 J. In some embodiments, the total energy delivered to the target site is equal to or greater than about 1000 J.

Non-steroidal anti-inflammatory drug (NSAIDs) use has raised concerns over possible dangerous complications such as GI bleeding, liver damage, and increased risk of major cardiovascular complications like stroke or heart attack. Individuals may be unable to take anti-inflammatory pills due to their unfavorable side effects. People are increasingly looking for alternate therapies and treatments. Studies have shown the use of offloading knee braces for treatment in knee osteoarthritis (OA) can relieve or reduce the pain. While the use of an offloading knee brace may help alleviate the pain and possibly slow the progression of knee OA it has not been shown to provide healing properties. A unique method to provide reduction in pain, better mobility, and healing of the knee OA is to combine the offloading brace with a light therapy device. The light therapy device (e.g., conformable flexible light emitting patch) is incorporated into the offloading knee braces, which provides support to the joint while the integrated light therapy device can provide automated treatment at the injury site. The light therapy treatment system 140 may be battery powered and microprocessor controlled to provide treatment at specified intervals to reduce inflammation and stimulate healing of the injury, as noted above.

It is noted that the light therapy treatment system 104 has a characteristic shape that conforms to the knee when it is bent at a specific angle. The light therapy treatment system 104 may be designed to accommodate an angle to accommodate optimum joint access. The conformable flexible light emitting system 122 is integrated into the light therapy treatment system 104 to provide light at a precise location. These concepts can be applied to other types of orthopedic appliances.

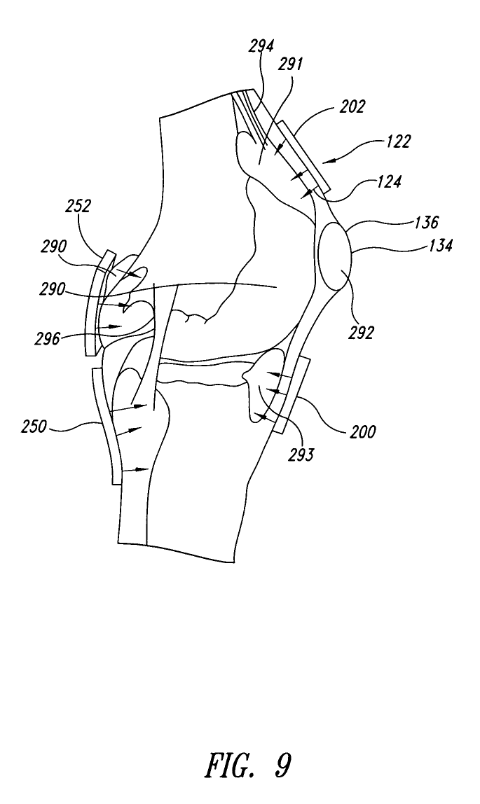

FIG. 9 shows the light emitting system 122 treating common target sites associated with osteoarthritis of the knee 134. The target sites, for example, can be synovial tissue containing the synovial fluid 290, is the “joint cavity,” and bursae 291, 292, 293. Synovial fluid helps lubricate and reduce friction forces while nourishing the articular cartilage. Folds in the synovial membrane 290 can form fluid-filled bursae 291, 292, 293 that function to lubricate adjacent soft tissue planes subject to yielding motion. As shown, light 124 can also be delivered to other tissues, such as the tendon 294 and/or ligament 296.

Traditional light therapy systems do not effectively delivery a therapeutic dose of light to these types of target sites because they merely emit a low density uniform light field. Thus, high intensity light is not aimed and delivered to specific target sites that cause or contribute to problems associated with arthritis or other unwanted conditions, diseases, or symptoms of the joints. The illustrated light emitting system 122 is positioned to delivery a therapeutically effectively amount of light to these important target sites to noticeably reduce symptoms associated with arthritis and may, in some instances, promote healing of the joint 136. The patches 200, 202 deliver light toward the infrapatellar and suprapatellar bursae 293, 291, respectively, and the joint cavity 290. The pair of posterior light emitting patches 250, 252 deliver light toward the joint cavity 290.

The light 124 shown in FIG. 9 is especially well positioned for treating bursitis. Bursitis occurs when one or more bursae become inflamed and cause pain during, for example, joint movement or direct pressure. As noted above, bursae are sacs filled with synovial fluid and function to provide cushioning between pressure points between mating bones and the muscles and tendons proximate the joint 136. A therapeutically effective amount of light 124 can be delivered to one or more inflamed bursae to reduce, limit, or substantially eliminate any inflammation. Other types of joints in the body can also be treated in a similar manner to delivery high intensity light to particular areas or regions of interest.

The light emitting system 122 can output energy at a high power density (e.g., a power density in the range of about 10 mW/cm² to about 200 mW/cm²). The system 122 may illuminate a large portion (e.g., a treatment area in the range of about 30 cm² to about 100 cm²) of the knee synovium 190, or other target site, if needed or desired. The total dose in the joint (including the synovium and surrounding tissue) can be in the range of about 200 J to about 3000 J. As such, a high therapeutic dose can be delivered to a large area to enhance overall effectiveness of the therapy.

Pulsing the light may improve the effectiveness of the therapy. In some embodiments, the light is pulsed in the range of about 10 Hz to about 100 Hz. In some embodiments, the light is pulsed at about 16 Hz. Other frequencies can also be used.

Light with more than one wavelength can be used concurrently, sequentially, or both during a therapy routine. The light 124 in FIG. 9 can include red light (e.g., a wavelength of about 620 nm-670 nm) and near infrared (e.g., a wavelength of about 820 nm-904 nm). Other wavelengths are also possible.

These parameter can also be used to treat other body parts. The total dose and treatment areas, however, can be adjusted in proportion to the target size (e.g., joint size) and target depth. For example, the total dose and treatment area for a hip joint may be substantially greater than the total dose and treatment area for a finger joint because of the relatively large size of the hip joint and its depth. Thus, the above parameters can be adjusted taking into account various factors, such as size of target area, depth to target area, optical properties of the tissue, and the like.

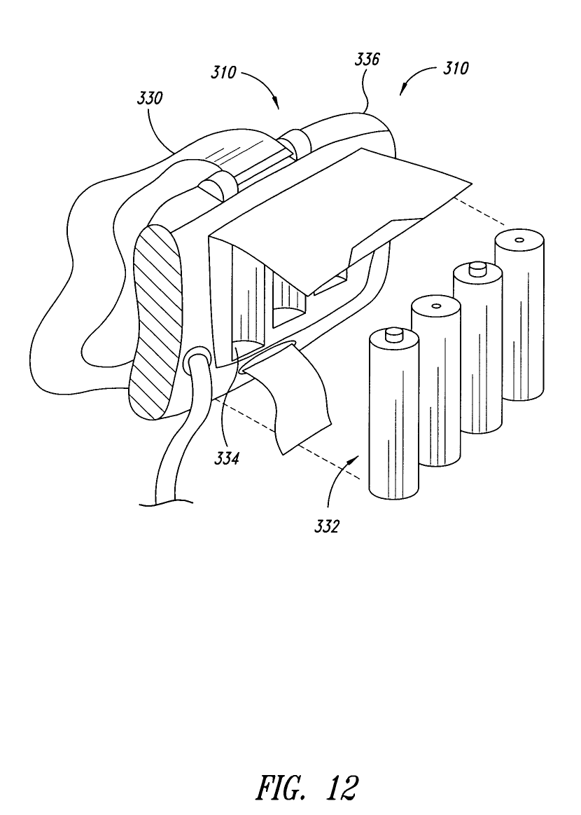

FIGS. 10A and 11 show a light therapy treatment system 300 usable to perform light therapy when the subject 100 is active, inactive, or both. The treatment system 300 includes a flexible light apparatus 304 the permits generally unrestrained movement of the leg 130. A pack 310 may be worn or carried by the subject 100 and is connected to a positioning structure 312 via a wireless or wired interface 314. A controller 315 is mounted on the pack 310. A pair of mounting systems 320, 322 holds a light emitting system against the individual's body part 130. In some embodiments, each mounting system 320, 322 holds at least one light emitting patch against the body part 130. The treatment system 300 may be generally similar to the light therapy treatment system 104 in FIGS. 1 and 2, except as further detailed below.

The power supply 310 and/or controller 315 may be removable from the pack 310 to allow easy replacement when not functioning or when a different control regime is desired, and/or to wash the pack 310. A power source 332 (FIG. 12) and/or controller 315 are coupleable to the conformable light apparatus 304 via the wireless or wired interface 314.

The wireless or wired interface 314 may provide power, communications and/or control for the conformable flexible light emitting system, and/or any sensors (e.g., temperature, moisture, light intensity, etc.) mounted to the light apparatus 304.

The treatment system 300 can be worn to perform ambulatory light therapy while the subject 100, for example, walks, runs, jogs, sits, sleeps, or performs other typical activities. Light therapy performed during movement can help facilitate light delivery to the target tissue by, for example, further distributing light, gaining greater coverage as compared to coverage in a generally static body part, adjusting (e.g., increasing and/or decreasing) the distance between the target tissue and the light energy source, and the like. When the illustrated knee 134 is moved, the synovial fluid in the knee may flow and therefore help distribute light to areas of inflamed tissue. Additionally, if a treatment agent is in the synovial fluid or other body fluid(s), movement of the knee 134 can promote movement and distribution of the treatment agent to increase the benefits of light therapy. Additionally or alternatively, moving tissue in the knee 134 can facilitate expansion of the internal treatment area, which in turn may facilitate illumination of the treatment area.

Clothing can conceal the treatment system 300. As such, the treatment system 300 can be worn at any time with minimal or an insignificant impact on normal daily life. For example, the treatment system 300 can be worn under a pair of pants without drawing attention to the user. In other embodiments, the treatment system 300 can be worn over clothing so that clothing does not have to be removed. Thus, light therapy can be conveniently performed any number of times throughout a day without altering or removing any clothing.

The pack 310 of FIGS. 10A to 12 can provide power to one or more components of the treatment system 300. The illustrated pack 310 is coupled to the leg 130 via a strap 330. As shown in FIG. 12, the pack 310 is configured to hold one or more batteries 332 that can be placed into a receiving chamber 334 in a main housing 336. The batteries 332 can be microbatteries, lithium ion batteries, polymeric battery, rechargeable or disposable batteries, super- or ultra-capacitors, or other types of batteries commonly used to provide electrical power. The batteries 332, for example, can be in the form of commonly used batteries for electronic devices (e.g., a personal data assistance (PDA), computer, cameras, music players (handheld music players such as MP3 players), and other portable electronic devices).

Alternatively, the treatment system 300 can be powered by an AC power source, such as a typical AC electrical power outlet. In some embodiments, the treatment system 300 is powered by an electric device (e.g., a PDA, computer, camera, music player, and the like) that can be used independently of the treatment system 300. For home or office uses, the treatment system 300 can have a connection configured to plug into port in a computer or other type of computing device, which can also function as a controller. Exemplary treatment systems 300 can have a cord (e.g., a USB cord) for connecting to a powered port (e.g., a USB powered port) of an electrical device, which outputs a sufficient amount of energy to power the light delivery apparatus 300 even if it is also operated to perform other tasks. Thus, various types of power sources can be used to power the treatment system 300.

The self-aligning light therapy treatment system 300, in some embodiments, can be a prophylactic brace, functional brace, or a rehabilitative brace. As used herein, the term “brace” is a broad term and may include, without limitation, a support that steadies or strengthens a portion of a subject's body. Braces can be flexible, semi-rigid, or rigid based on their intended function. In some embodiments, the brace can allow substantial joint motion (e.g., joint articulation) during therapy. Where the brace is a knee brace, the user may be able to run, jog, walk, or perform other normal activities. Such braces can be hinged and may provide support (e.g., lateral support), stabilize the kneecap, and the like. FIG. 10B shows a treatment system having a movable hinge 317 for permitting substantial joint motion. The amount of joint motion can be selected based on the light therapy to be performed.

The prophylactic brace 300 can have one or more light emitting sources or patches that deliver light to target sites commonly injured during sporting activities, such as football. The prophylactic brace 300 can both reduce the likelihood of an injury and provide light therapy. The functional brace 300 can have one or more light sources or patches that deliver light to target tissues that the brace 300 is designed to help (e.g., joint tissue that the brace 300 is designed to protect). Rehabilitative braces can have one or more light sources or patches that deliver light to injured tissue or a surgery site. The rehabilitative brace 300 can both provide mechanical support to promote repairing of tissue and provide light therapy. The rehabilitative brace 300 can limit harmful knee movement while the light is delivered to the injured or damage tissue to accelerate the healing process, manage pain, and the like. Unlike traditional light therapy devices (e.g., light pads), these types of braces can improve joint functioning (e.g., joint mechanics), protect the joint, and/or bear loads to facilitate healing, as well as performing other functions known in the art. The braces can be in the form of a knee brace (described above), wrist brace, elbow brace, ankle brace, shoulder brace, back brace (e.g., a lower back brace), ankle brace, finger brace, toe brace, hip brace, jaw brace, and the like.

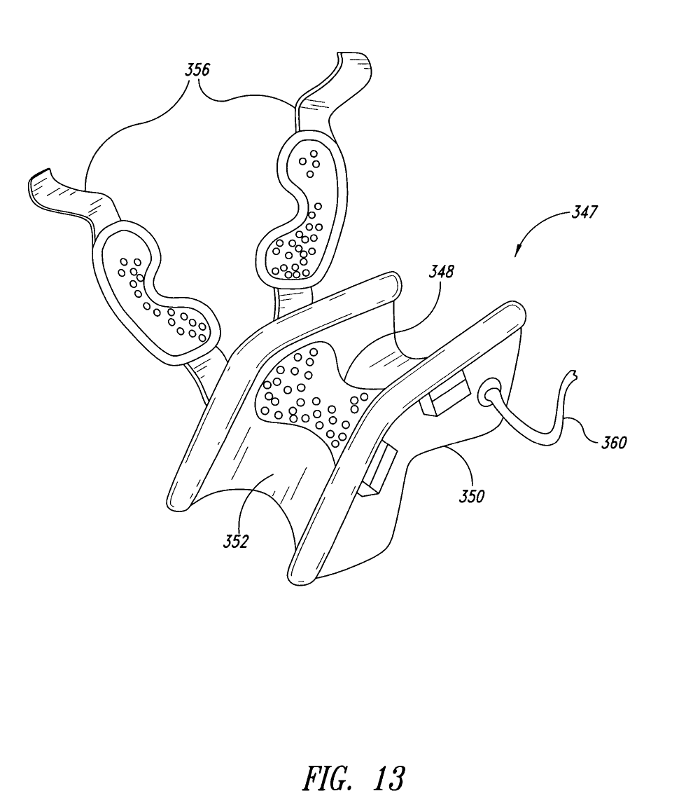

FIG. 13 shows a treatment system 347 including a conformable flexible light emitting patch 348 carried by a positioning structure in the form of a brace 350. The brace 350 includes a substrate 352. The brace 350 also includes one or more straps 356 for securing the brace to a treatment site.

The brace 350 may further include a wireless or wired interface 360 to provide power, communications and/or control for the conformable flexible light emitting patch 348 and/or any sensors (e.g., temperature, moisture, light intensity, etc.) mounted to the brace 350.

Various components of the treatment systems described above can be incorporated into other types of braces. Light emitting systems, light sources, light arrays, patches, controllers, and other components disclosed herein can be coupled to or incorporated into traditional braces. The positions of the light sources or emitting systems can be selected based on the target treatment area. For example, the light emitting system shown in FIG. 6 can be incorporated into the braces disclosed in U.S. Pat. Nos. 5,797,864; 5,400,806; and 5,562,605, which are incorporated by reference in their entireties. These braces may be modified by one of ordinary skill in the art based on the desired size, location, and geometry of the light sources or systems.

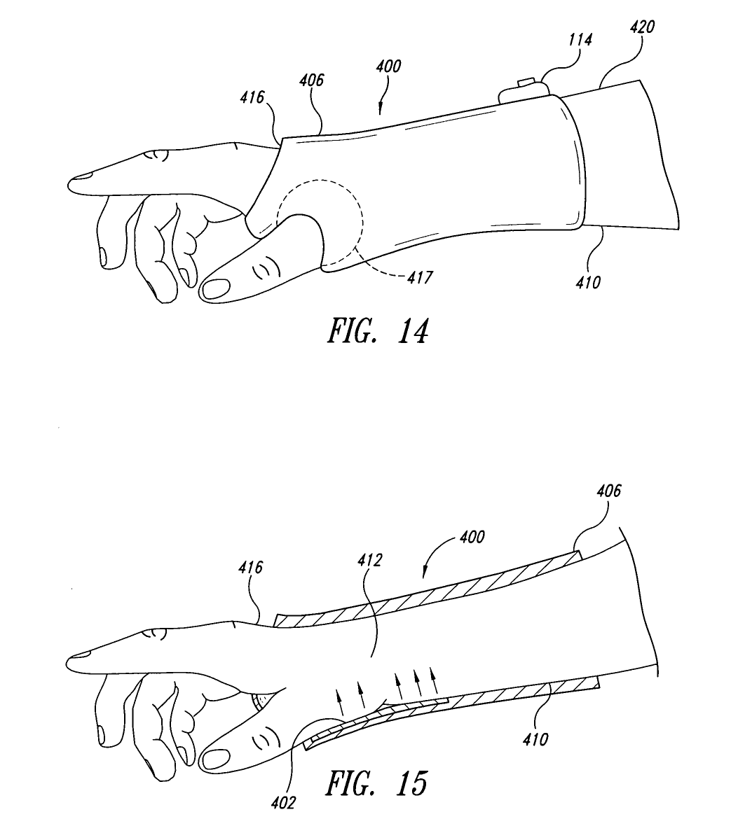

FIGS. 14-16, 18-21, 23-24, 27-29, and 31-36 show other types of orthopedic appliances for performing light therapy. Each of these orthopedic appliances may be generally similar to the treatment systems described above, except as further detailed below.

FIG. 14 illustrates a light therapy treatment system for placement over a wrist. Typically the wrist is positioned in slight dorsiflexion. The illustrated light delivery system 400 is in the form of a wrist brace for treating carpal tunnel syndrome, but the wrist brace 400 can be configured to treat other conditions or diseases as well.

The illustrated wrist brace 400 includes a light emitting system 402 in the form of a light patch and wearable positioning structure 406 extending along the forearm 410, the wrist 412, and partially surrounding the hand 416. The emitting system 402 is sandwiched between the subject and the positioning structure 406.

The light delivery system 400 is positioned to deliver light to tissue that can reduce, limit, or substantially eliminate pain or discomfort associated with carpal tunnel syndrome. For example, the light delivery system 400 can target tissue that directly or indirectly causes pressure on the median nerve in the wrist 412. The median nerve enters the hand 416 by passing through the carpal tunnel formed by the carpal bones and transverse carpal ligament in the wrist 412. Light therapy can be performed on the median nerve and tissue (e.g., inflamed tissue) near or adjacent the median nerve. In this manner, the pressure on the median nerve can be reduced to treat painful throbbing, numbness, and/or tingling sensations in the hand 416, wrist 412, and/or arm 420 which are often experienced with carpal tunnel syndrome. The light delivery system 400 can be modified to treat other conditions or diseases. For example, the system 400 can perform light therapy on the 1^{st} CMC joint 417 (or other the carpal metacarpal joints).

The positioning structure 406 can be a flexible, semi-rigid, or rigid shell designed to closely surround the arm 420, forearm 410, wrist 412, and hand 416. The mechanical function of the positioning structure 406 can be selected base on whether the wrist brace is a prophylactic brace, functional brace, or a rehabilitative brace. In some embodiments, for example, the wrist brace 400 can generally fix the wrist 412 in a desired position suitable for treating carpal tunnel syndrome and performing light therapy. Light emitting systems disclosed herein can also be incorporated into commercially available wrist braces used to treat a variety of conditions.

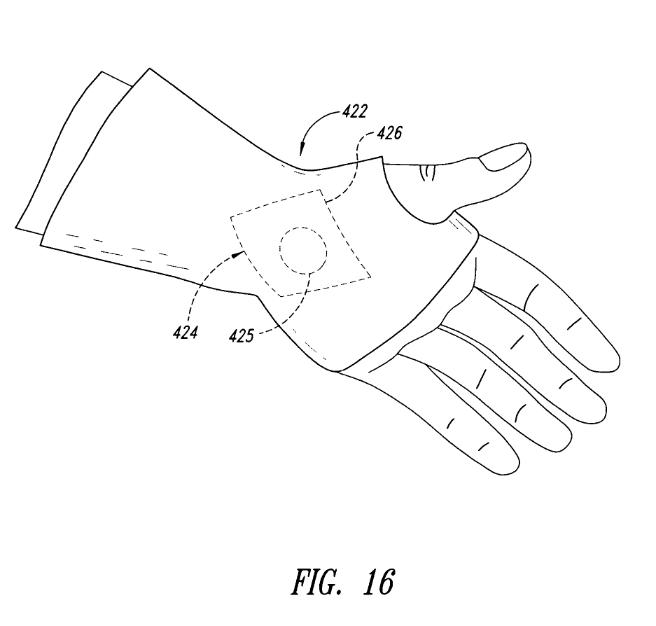

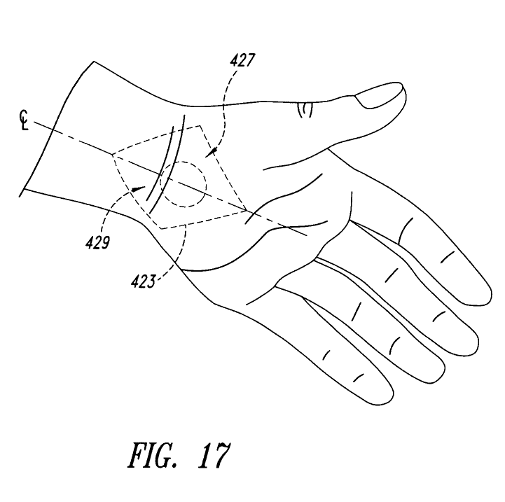

FIG. 16 shows a light therapy treatment system 422 that provides light therapy to regions often inflamed due to repetitive stress injuries, such as carpal tunnel syndrome. The illustrated treatment system 422 includes a light emitting system 424 (shown in phantom in FIG. 16) having a plurality of emitting regions for independently emitting light. The illustrated light emitting system 424 has a first emitting region 425 for treating a first target treatment area 423 (FIG. 17) and a second emitting region 426 for treating a second treatment area 427 (FIG. 17). The first emitting region 425 can provide high intensity light therapy to the first treatment area 423, and the second emitting region 426 can provide low intensity light therapy to the second treatment area 427, or vice versa.

The number, sizes, and locations of the target treatment areas may vary between subjects, or between conditions or diseases. The illustrated treatment area 427 surrounds the treatment area 423 and is positioned generally along the centerline CL of the wrist. The treatment area 427 extends across and distally of the flexor crease 429. Because the treatment areas 423, 427 are proximate the wrist joint, the wrist flexor crease 429 is an anatomical feature suitable for locating and aligning the system 422.

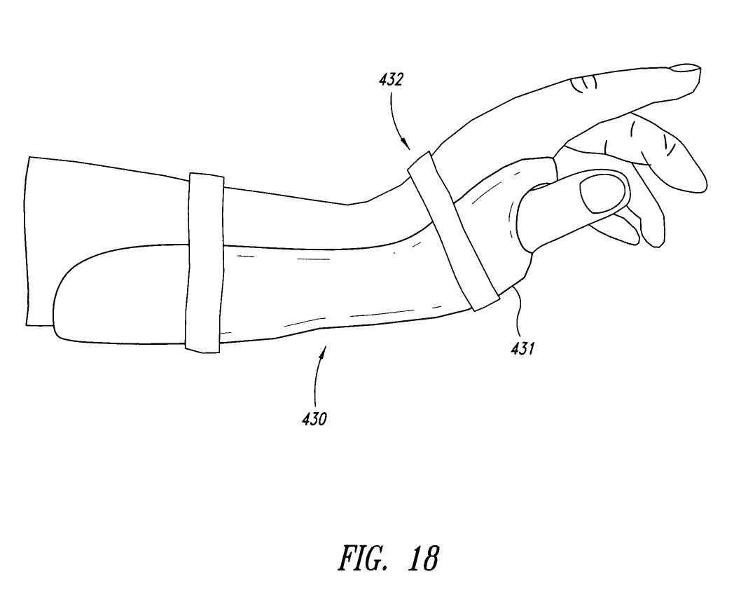

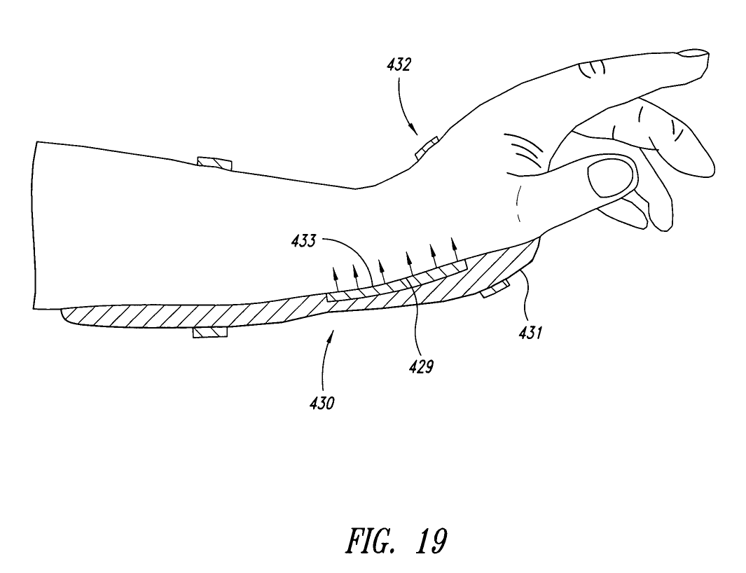

FIGS. 18 and 19 show a light therapy treatment system 430 configured to position the wrist for improved light delivery. The illustrated treatment system 430 maintains dorsiflexion of the wrist and includes a positioning structure 431 that is coupled to the wrist via a mounting system 432. As shown in FIG. 19, a light emitting system 433 is positioned adjacent the flexor crease 429. More light may reach the joint tissue as compared to the amount of light that reaches the joint tissue with the wrist in a generally straight position.

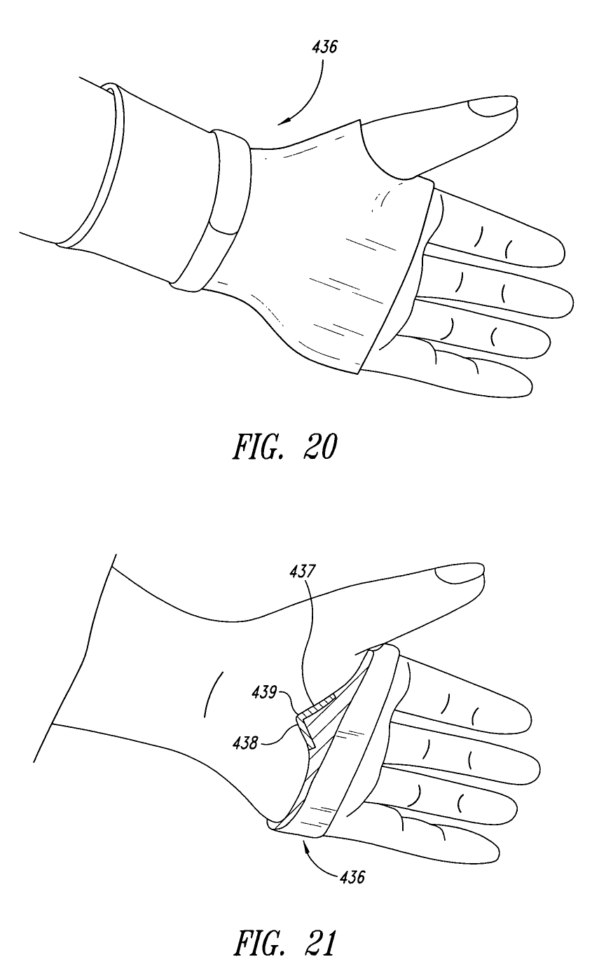

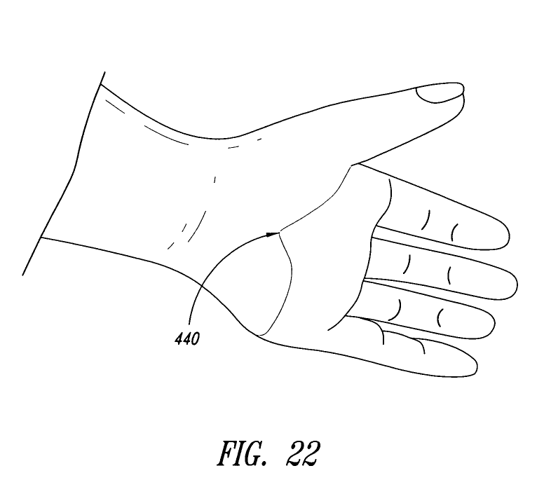

Other anatomical features can be used to locate light therapy treatment systems for providing light therapy to the wrist, hand, and/or forearm. For example, FIG. 20 shows a light therapy treatment system 436 that aligns itself using the thenar and/or hypothenar muscles. As shown in the cut-away view of FIG. 21, the treatment system 436 has a positioning structure 437 having a protruding locator 438 dimensioned for placement at the junction 440 (see FIG. 22) of the thenar and hypothenar muscles. In the illustrated embodiment, the light emitting system 439 forms at least a portion of the locator 438.

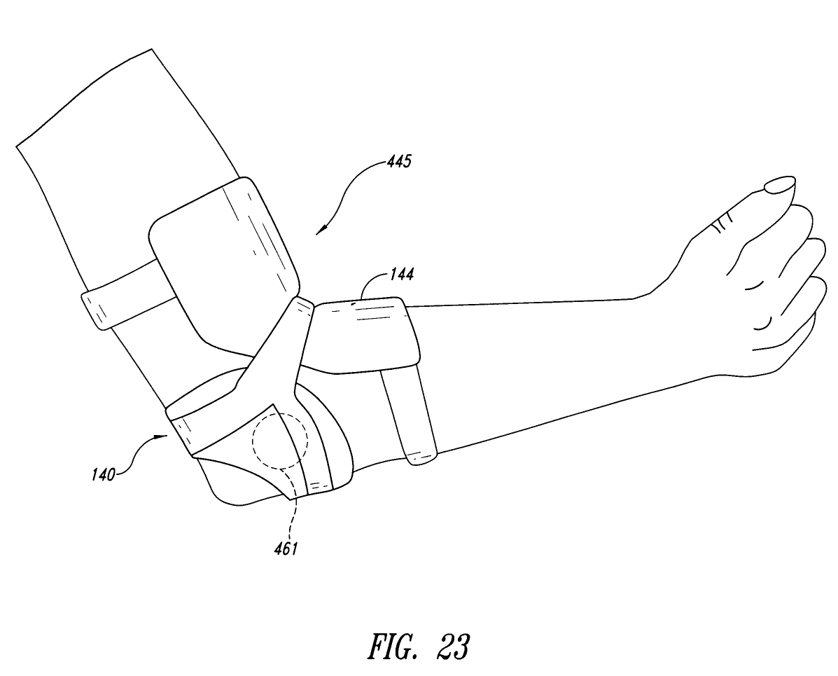

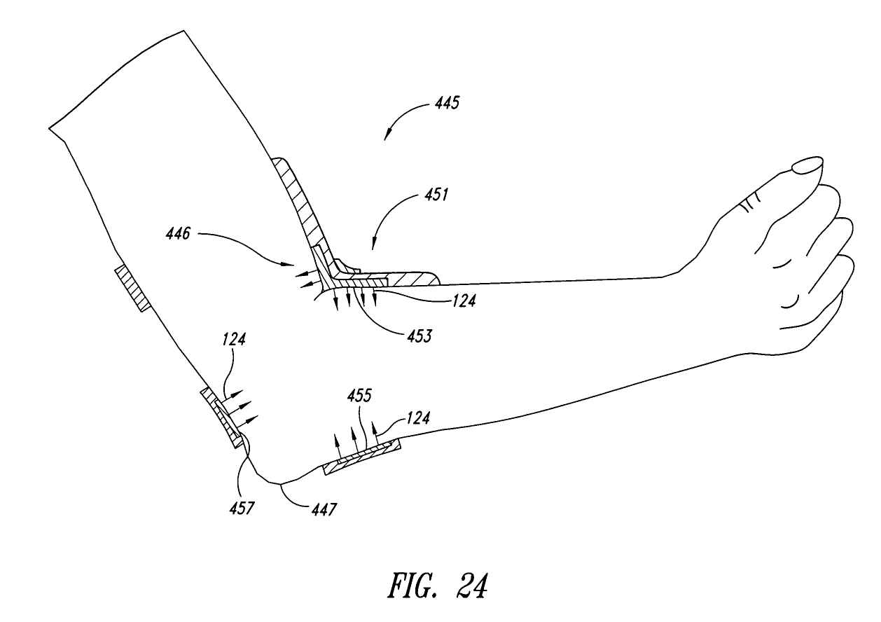

FIG. 23 shows a light therapy treatment system 445 for providing light therapy to the elbow. The treatment system 445 has a light emitting system 446 (see FIG. 24) positioned to treat the elbow joint 447.

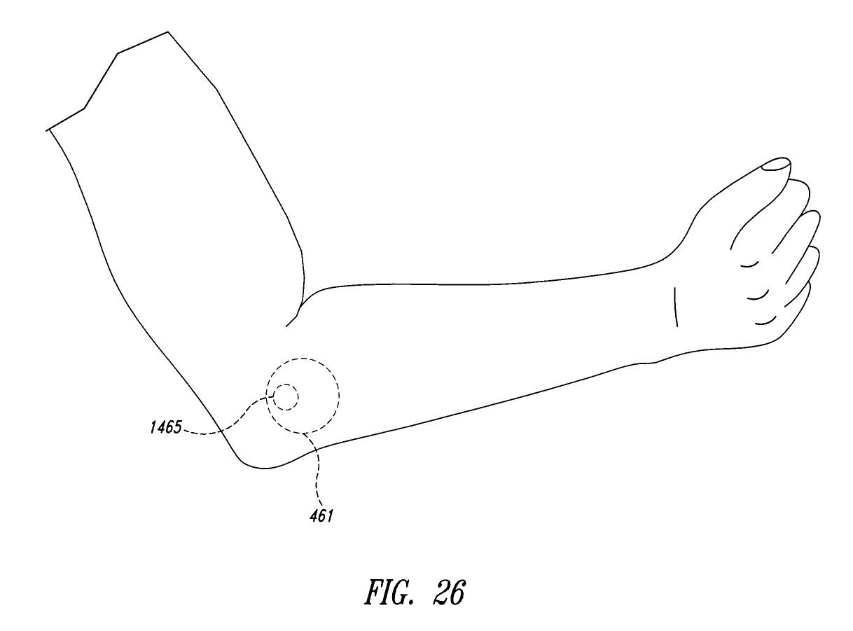

Referring to FIG. 24, the light emitting system 446 includes patches 453, 455, and 457. The patch 453 can extend across the joint crease. In some embodiments, one or both of the patches 455, 457 can be positioned to treat the target treatment area 461 (shown in phantom in FIGS. 23 and 26). In some embodiments, a separate patch can provide light therapy to the target treatment area 461.

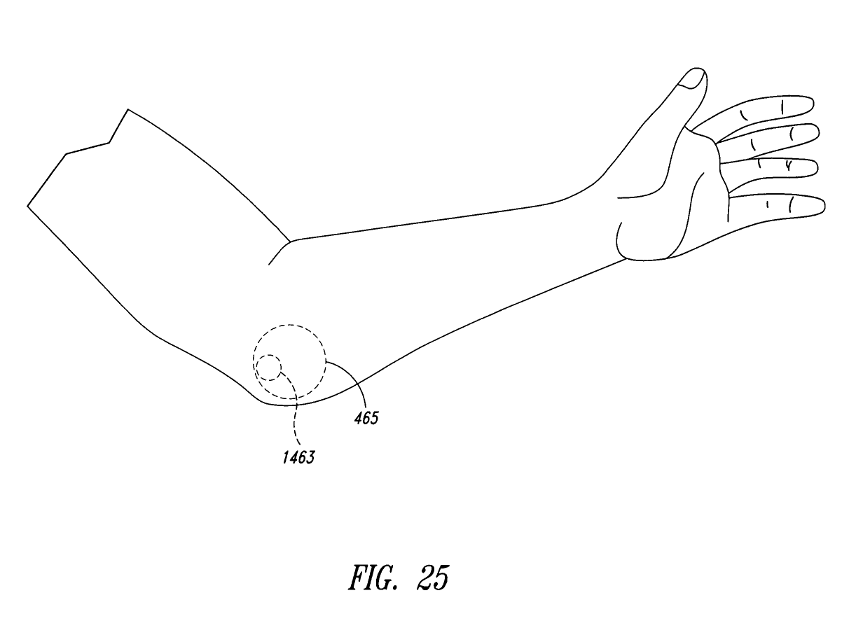

With respect to FIG. 25, to position the treatment system 445, the mounting system 140 can cover and engage the medial epicondyle 1463 to treat the target site 465. In other embodiments, the mounting system 140 can cover and engage the lateral epicondyle 1465 (see FIG. 26). Such embodiments are well suited to provide light therapy at a treatment site 461 at least proximate the lateral epicondyle 1465.

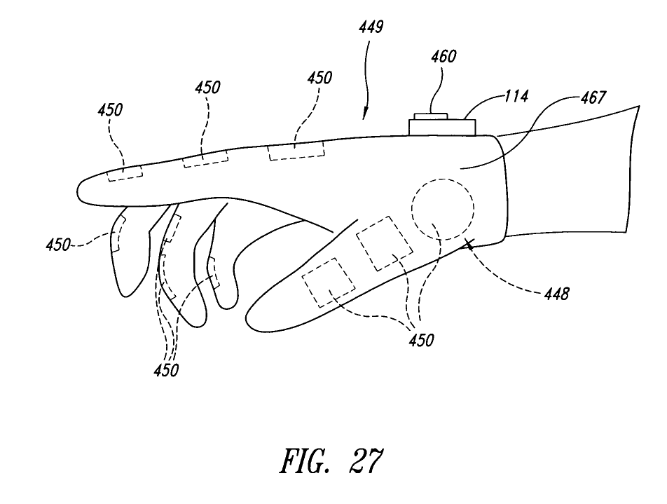

FIG. 27 shows a light therapy treatment system 449 used to treat a hand. The illustrated system 449 is in the form of a glove having a light emitting system 448 having a plurality of light emitting patches 450 (shown in phantom) positioned to treat joints of the hand. The illustrated emitting patches 450 are coupled to an interior surface of a glove main body 467. A power supply 460 can provide power to the light emitting system 450. The controller 114 is used to control the operation of the light emitting system 447.

Various joints, including, without limitation, metacarpophalangeal joints, carpal-metacarpal (CMC), proximal interphalangeal joints, and distal interphalangeal joints, can be treated with the system 440. For example, the system 440 can be programmed to treat any joint (including wrist, thumb or finger joints) causing pain or discomfort. For example, the light emitting system 448 is well suited to treat finger or thumb arthritis, including osteoarthritis. In some embodiments, light emitting systems 450 each comprise one or more patches for conformally engaging corresponding joints (such as the 1^{st} CMC joint) while providing a comfortable fit.

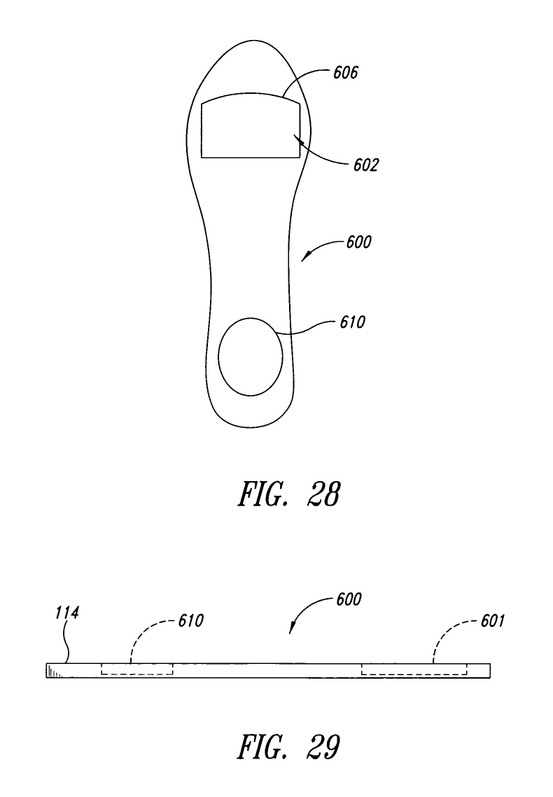

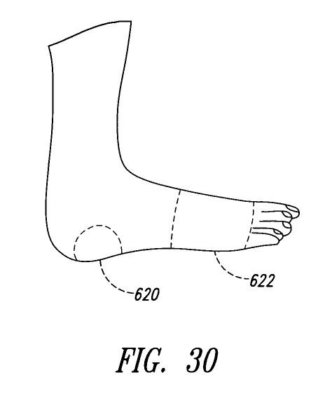

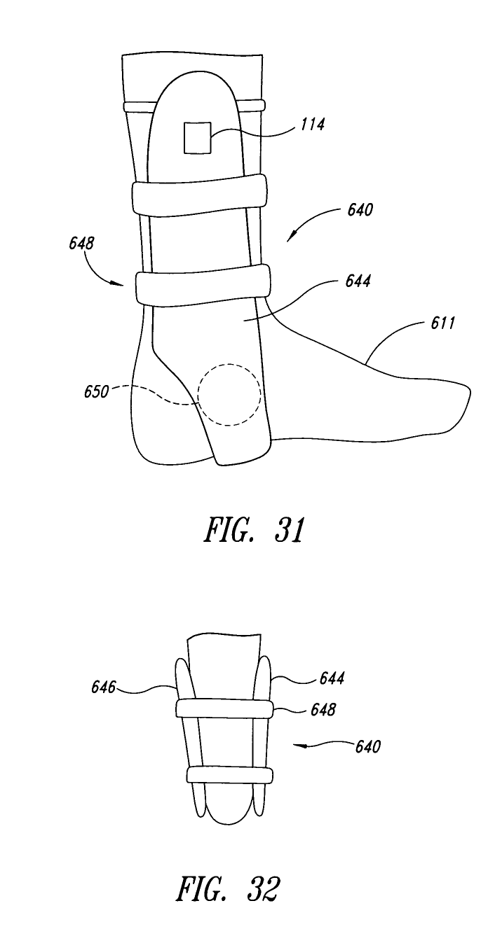

Light emitting systems can also be incorporate into other types of garments, clothing, footwear, or orthopedic appliances. For example, light delivery systems can be incorporated into socks, shoes, or other footwear to target tissue in the foot. FIGS. 28 and 29 show a light therapy treatment system 600 in the form of an insole for placement in footwear (e.g., a shoe, boot, etc.) or a sock (see, e.g., a sock 611 in FIG. 31) and includes a light system 602 suitable for standing upon. The light system 602 includes a pair light emitting patches 606, 610 positioned to treat the metatarsalgia 622 and plantar fascitis 620, respectively (see FIG. 30). The systems can deliver light to the bottom and sides of the subject's foot. In other embodiments, the treatment system 600 can be placed on top of the foot to treat, for example, the metatarsal heads. The thin layer of skin on the top of the foot provides efficient delivery of light to the metatarsal heads or other internal tissue. In some embodiments, the treatment system 600 can be incorporated into the sock 611 (FIG. 31) or other footwear. One or more treatment systems 600 in a sock can be well suited for treating bunion pain, dorsal foot pain, or other conditions. For more severe sprains or injuries the entire foot and ankle can be treated.

FIGS. 31 and 32 show a light therapy system for treating an ankle. The illustrated system 640 includes a pair of positioning structures 644, 646 and a mounting system 648. The positioning structures 644, 646 can mate with various anatomical features (e.g., the lateral malleolus, heel or calcaneus, arch, etc.) to provide alignment. A light emitting system 650 (shown in phantom in FIG. 31) can provide light therapy to the lateral malleolus (often the primary target to which high levels of light energy can be delivered), anterior talofibular ligament, plantar facia attachment, or other locations of interest. The configuration and dimensions of the brace can be selected based on the target site(s).

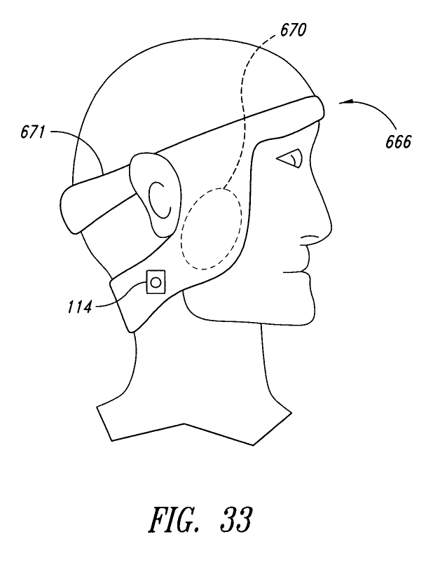

With respect to FIG. 33, a light therapy treatment system can be configured for treating a treatment site at the head. The illustrated system 666 is a headset configured to treat the palpable temporomandibular joint (TMJ), and may function as a jaw brace, if needed or desired. The light emitting system 670 is coupled to a main body 671. The main body 671 is sized to surround and engage the ear, which serves as a locator. Light emitting systems can also be mounted to other types of helmets, headsets, or jaw braces, which may use the ear, angle of jaw (mandible), chin, and/or other anatomical features as locators.

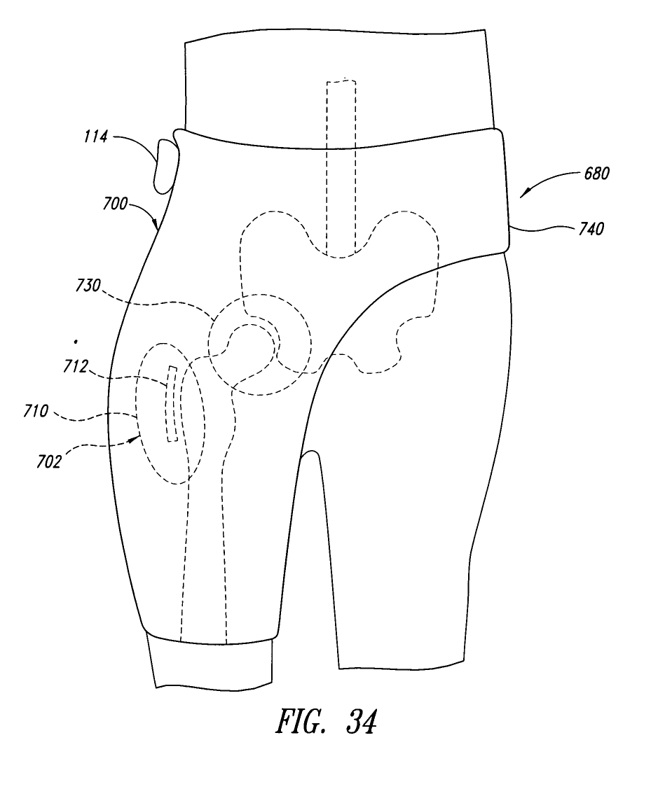

FIG. 34 shows a light therapy treatment system 700 for delivering light therapy to the hip joint. The system 700 includes a light emitting system 702 having a patch 710 for delivering light energy to the greater trochanteric bursa 712 or other bursa in the hip. Additionally or alternatively, the emitting system 702 can have a patch 730 for delivering light energy to the hip joint.

The system 700 may include a flexible or semi-flexible main body 740 to allow joint motion. The light emitting system 702 can be adhered, bonded, mechanically coupled, or otherwise attached to an interior surface of the main body 740. In other embodiments, the light emitting system 702 is incorporated into the main body 740 itself. A variety of main bodies can be used to hold the light emitting system 702 in the desired position. The anterior superior iliac spine, greater trochanter palpable, and other features can be used as locators.

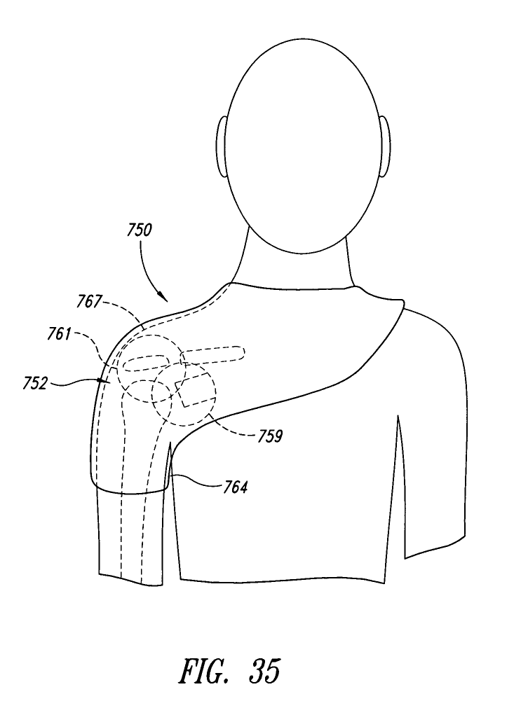

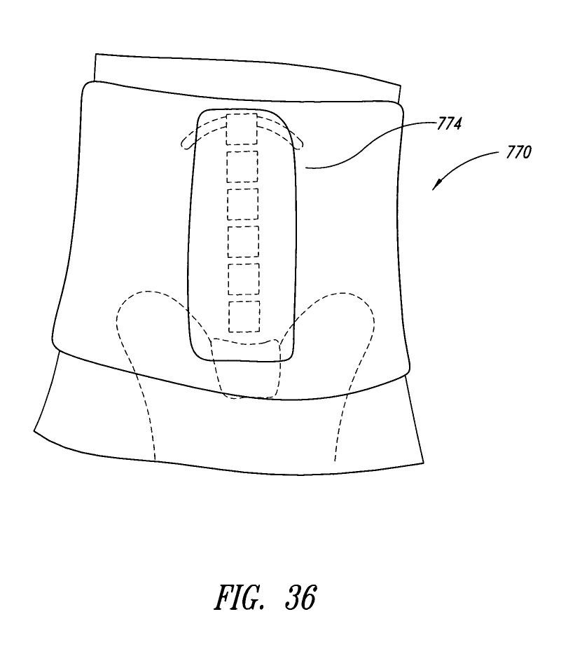

FIGS. 35 and 36 illustrate a shoulder brace and back brace, respectively, that are generally similar to the braces described above. The shoulder brace 750 of FIG. 35 has a light emitting system 752 for providing light therapy to the shoulder joint (e.g., the acromion, subacromial bursa, rotator cuff, acromioclavicular joint, glenohumeral joint, and the like). The illustrated light emitting system 752 includes patches 759, 761. The deltoid 762, axialla 764, or other features can be used as locators. Auxiliary armpit patches can be provided to treat the glenohumeral joint or other portions of the subject's body.

Referring to FIG. 36, the light therapy treatment system 770 is in the form of a back brace that includes a light emitting system 774 positioned to provide light therapy on the spine. The illustrated emitting system 774 is an elongate patch that extends generally along the longitudinal axis of the spine. The patch 774 extends laterally from the spine to ensure that a somewhat uniform light field is delivered to various locations of along the spine, including sacroiliac joints.

Light therapy can be used to treat various types of back conditions that often lead to pain or discomfort. The midline (spinus processes), anterior superior iliac spine, posterior superior iliac spine, sacrum (sacral spine), and other features can be used to locate the brace 770. The brace 770 can include depressions or recessed regions that mate with anatomical feature(s) functioning as locators.

FIG. 5B shows a multi-modality light therapy treatment system 500 that provides one or more modalities of treatment procedures, such as light therapy and another type of therapy (e.g., a non-light penetrating energy therapy). The illustrated light therapy treatment system 500 may be generally similar to the light therapy treatment system 104 illustrated in FIG. 1, except as further detailed below.

The light therapy treatment system 500 includes a light emitting system 122 and a non-light energy delivery system 506 for performing a secondary non-light energy therapy. For example, the non-light energy delivery system 506 can deliver a therapeutically synergistic amount of non-light energy to the target site such that the combination of light energy therapy and non-light energy therapy results in increased beneficial physiological effects as compared either light therapy or non-light energy therapy used alone. In some embodiments, the increased overall beneficial physiological effects are substantially greater than the beneficial physiological effects obtained by either light therapy or non-light energy therapy used alone.

Beneficial physiological effects may include, without limitation, reduction of pain, rate of healing, reduction of inflammation, and the like. Non-light energy therapy is broadly construed to include, but is not limited to, ultrasound therapy, microwave therapy, radiofrequency therapy, mechanical therapy, electro-magnetic therapy, electrical therapy (e.g., low level electrical current therapy), and the like. The non-light energy delivery system 506 can include, without limitation, one or more transducers, such as acoustic transducers, ultrasound transducers, magnetic transducers, electro-magnetic transducers, pressure transducers (e.g., mechanical impulse transducers), and other types of transducers suitable for use on a subject. The transducers can be energized to output penetrating energy that causes cell stimulation or activation. The non-light energy delivery system 506, in some embodiments, may be a field generator (e.g., an electro-magnetic field generator), radiofrequency emitter, vibrator (e.g., an unbalanced mass vibration system), electrical stimulator (e.g., electrical stimulators configured selectively output low levels to high levels of electrical currents), and the like.

The non-light energy delivery system 506 of FIG. 5B includes a plurality of non-light energy delivery devices 510, 512, 514, 516, 518, 520 positioned to deliver energy at or near the target sites targeted by the light emitting system 122. That is, both the non-light energy delivery system 506 and light emitting system 122 can output energy in the same general direction and, consequently, can cooperate to achieve the desired physiological effects at specific target sites. In some embodiments, the non-light energy delivery system 506 is positioned in proximity to the light emitting system 122 to help ensure proper alignment of their outputted energies. In other embodiments, the non-light energy delivery system 506 is spaced a substantial distance from the light emitting system 122 such that their outputted energies are transmitted along different delivery paths through the patient but may still reach the target sites. Such embodiments may reduce or substantially eliminate concurrent treatment of intermediate tissues, e.g., intermediate tissue between the target site and the non-light energy delivery system 506 or intermediate tissue between the target site and the light emitting system 122, often leading to unwanted collateral treatment.

The number and placement of the non-light energy delivery devices 510, 512, 514, 516, 518, 520 can be chosen based on the desired synergistic interaction between the light therapy and the non-light energy therapy. Additionally, various types of non-light energy delivery devices can be incorporated into the light therapy treatment system 500 to provide any combination of ultrasound therapy, microwave therapy, radiofrequency therapy, mechanical therapy, vibration therapy, pressure therapy, electro-magnetic therapy, and electrical therapy. Accordingly, a single light therapy treatment system 500 can be used to perform a wide range of specialized treatment programs.

The energy from the non-light energy delivery system 506 can be at various intensities, frequencies, wave forms (e.g., square waves, triangle waves, sinusoidal waves, saw-tooth waves, and/or square waves), square wave pulse trains, trigometric wave pulse trains, sinusoidal wave pulse trains, square wave pulse trains, and other types of wave trains suitable for treating a subject.

The devices 510, 512, 514, 516, 518, 520 can be fixed or variable mode devices depending on the treatment procedure. Thermal devices 510, 512, 514, 516, 518, 520, such as resistive heaters, can operate on a fixed power mode, whereas acoustic devices 510, 512, 514, 516, 518, 520 can operate on a variable frequency to perform therapies at a variety of frequencies.

To perform acoustic therapy, the devices 510, 512, 514, 516, 518, 520 in the form of acoustic transducers can output acoustic energy at a frequency between about 10 kHz and about 20 MHz. For example, in one embodiment, the acoustic waves have a frequency between about 200 kHz and about 20 MHz. In another embodiment, the waves have a frequency between about 1 MHz and about 3 MHz. In yet another embodiment, the waves have a frequency of about 2 MHz. The average acoustic power can be between about 0.1 watts and 400 watts. In some embodiments, the average acoustic power is about 15 watts.

To enhance delivery of energy, a transmission media can be applied to the skin. The transmission media can increase the amount of energy reaching the skin, thus increasing the amount of light ultimately reaching the target site. This can increase the rate of energy delivery (thereby shortening the treatment period) and the total amount of energy that ultimately reaches the target site possibly improving the efficacy of the therapy session. Transmission media can include, in some embodiments, one or more coupling fluids or gels that facilitate propagation of energy to the patient.

Transmission media can be a gel, such as an optical clearing gel (e.g., glycerin gel), suitable for placement between the light emitting system 122 and the subject. Other types of transmission media can also be used. For example, transmission media can be designed to transmit non-light energy to the tissue. An acoustic coupling media (e.g., a coupling agent or gel) can be used to ensure good acoustic coupling between an acoustic transducer and the treatment site. Additionally, water, saline, water-based solutions, ultrasound gels or any other suitable transmission media can be used in combination with the transducers and light sources disclosed herein. The transmission media can be spread before and/or during the therapy session. It is contemplated that one or more layers of acoustic coupling gel can be disposed between the patient and any light energy source and/or the patient and any non-light energy source.

Non-light therapy can result in more consistent and faster beneficial responses in a subject than using light therapy alone because of non-light therapy operating on the same or different physiological features of the patient's body. For example, light therapy and non-light therapy can operate on different cellular pathways, and/or different physiological pathways. As such, complementary light therapy and non-light therapy can be selected to affect different physiological features of the subject's body providing enhanced flexibility when determining an appropriate treatment protocol.

Additionally or alternatively, light therapy can prepare target sites for subsequently performed non-light therapy. In some embodiments, for example, light therapy can predispose the target sites to a desired physiological response when subjected to the non-light therapy. Conversely, non-light therapy can prepare one or more target sites for subsequently performed light therapy.

Various types of non-light energy delivery systems can also incorporated into the light therapy treatment systems illustrated in FIGS. 1-5A, 10A-11, 13, 14-16, 18-21, 23, 27-29, and 31-36, as well as other orthopedic appliances disclosed herein.

FIG. 5C shows a light therapy system having a plurality of detectors 527 that are communicatively coupled to the controller 114 or other type of control system. The detectors 527 (e.g., temperature detectors or sensors, optical detectors, pressure sensors, or other types of sensors or input devices) are configured to measure at least one physiological indicator.

The controller 114 determines at least one operating parameter (e.g., power density, treatment type, treatment duration or period, depth of penetration, pulse intensity, pulse duration, pulse repetition rate, stop-start time, position and orientation of the light emitting system, and the like) based at least in part on a signal from at least one of the detectors, wherein the signal is indicative of the physiological indicator.

The detectors can be used to ensure proper treatment and prevent excess illumination, overheating, and the like. If the light emitting system generates appreciable amounts of heat, the detectors 527 of FIG. 5C can be temperature sensors. The detectors 527 can ensure that the temperature of the subject's skin is maintained at an acceptable level. In some embodiments, the detectors 527 can comprise, without limitation, one or more temperature sensors, thermocouples, pyrometers, and the like.

Additionally or alternatively, the detectors 527 can be optical sensors used to monitor the amount of light delivered to the target site, the appearance of the tissue (e.g., color of the skin), or other measurable optical characteristics. Using signals from the optical detectors 527, the controller 114 can determine appropriate treatment parameters. The optical detectors 527 may comprise filters, charged coupled detectors (CCD), mercury-cadmium-telluride (MCT) detectors, and the like. Any number of detectors can be used, including any detector type suitable for sensing electromagnetic energy, such as infrared energy.

In some embodiments, one or more pressure sensors can be utilized to ensure proper treatment. A pressure sensor can measure the pressure applied to the patient, and may be used to determine whether the light emitting system properly engages the subject. If the controller 114 determines that the applied pressure is at or below a threshold pressure (e.g., a light patch may not be properly contacting the subject), the controller 114 can alert the user and/or stop the light delivery process. These types of sensors can also be used to determine the size and geometry of the body part.

In yet other embodiments, the detectors 527 can be used to determine the composition of the body tissue. For example, the detectors 527 can measure the resistance of the body tissue to determine the body fat percentages. Various combinations of detectors, sensors, timers, and the like can be used to ensure proper treatment of the patient.

The controller 114 can have a closed loop or open loop system. For example, the control system 114 can have a closed loop system, whereby the power to the light emitting system is controlled based upon feedback signals from one or more sensors configured to detect and transmit (or send) one or more signals indicative of temperature, pressure, optical properties, composition of tissue (e.g., body fat percentage at target site), size of target site (e.g., large target site vs. small target site), size of body part, or any other measurable parameters of interest.

Based on those readings, the controller 114 can then adjust the output from the light emitting system. Alternatively, the system 500 can be an open loop system wherein the amount of stimulation produced by the light emitting system 144 is set by user input. For example, the light emitting system may be set to a fixed power mode by utilizing the controller 114. It is contemplated that the system 500 can be switched between a closed and open loop system. One or more of the detectors 527 can be incorporated into the other treatment systems disclosed herein.

Various concepts of the embodiments disclosed below (including electrical circuitry) can be incorporated into the embodiments described above for enhanced performance. As used herein, “panel” is a broad term and may include, without limitation, a patch or blanket having an array of light sources, but more generally includes any flexible light emitting system for providing light therapy.

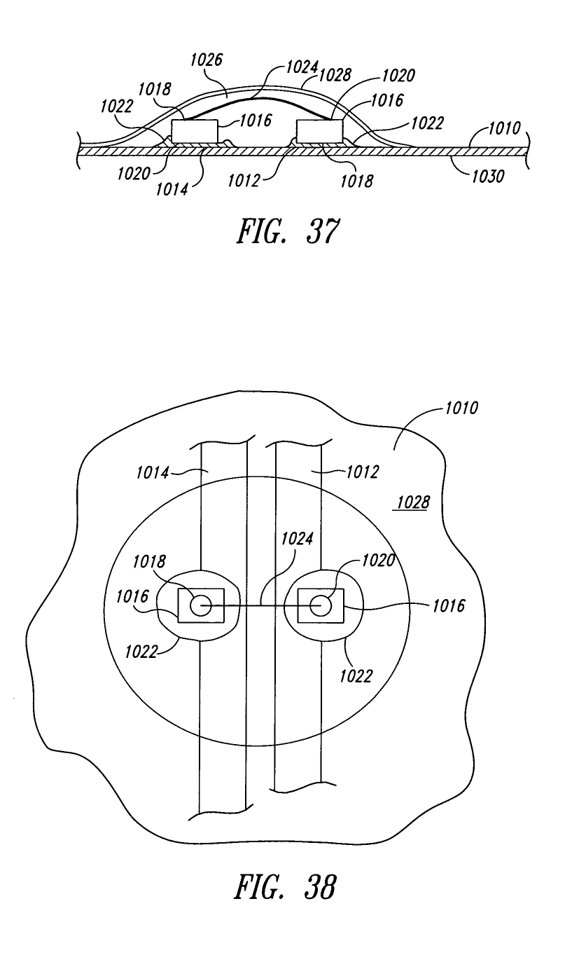

In FIG. 37, a small portion of a flexible substrate 1010 is illustrated that is used in creating a conformal flexible light emitting patch adapted to provide a close fit over a non-planar portion of a subject's body for treating external or subcutaneous abnormal tissue at that treatment site by administering light therapy. Advantageously, the conformal flexible light emitting patch can generally match the geometry of the subject to which it is applied. In some embodiments, small spaces may be formed between the conformal flexible light emitting patch and the subject's body, but the patch may still delivery an effective dose of light energy to the target site. The closeness of the fit can be selected based on the desired light transmission efficiency.

Additional details that disclose how flexible substrate 1010 is able to more readily conform to irregularly shaped portions of the subject's body to provide a close fit are disclosed below. Flexible substrate 1010 may, for example, be less than 0.1 millimeter thick and may be fabricated from a highly flexible thin film polymer such as silicone or polyurethane.

Conductive traces 1012 and 1014 are formed on a surface of flexible substrate 1010 that is adapted to face toward a treatment site on the subject's body to which light therapy is to be administered. These conductive traces may, for example, be formed using a conductive ink applied in a liquid form and allowed to set, or some other extremely flexible conductive media. Conductive ink works well for this purpose, since it produces a very thin conductive trace after it dries and is readily applied in any desired configuration to form an electrical circuit on the surface of the flexible substrate.

FIG. 38 illustrates portions of electrical traces 1012 and 1014 that extend generally parallel to each other. The traces 1012, 1014 are spaced apart sufficiently to enable two light emitting sources 1016 to be mounted on the flexible substrate between the electrical traces and each in electrical contact with one of the electrical traces. Light emitting sources 1016 may, for example, each comprise a broad spectrum light source such as an incandescent, halogen, fluorescent, or electroluminescent light source, or may comprise either a light emitting diode (LED) or a specialized type of LED, such as a polymeric, an organic, or a metallic LED.

As illustrated in FIGS. 37 and 38, light emitting sources 1016 are electrically mounted on conductive trace 1012 and conductive trace 1014 using a conductive bonding adhesive 1022, which is applied to the conductive trace to secure one side of light emitting source 1016 to that conductive trace. In the embodiment disclosed in FIGS. 37 and 38, light emitting sources 1016 are mounted as pairs disposed adjacent each other, with one light emitting source of the pair being adhesively attached to conductive trace 1012, and the other adhesively attached to conductive trace 1014 using conductive adhesive 1022. An anode 1018 of one of the light emitting sources is electrically coupled to conductive trace 1012, while a cathode 1020 of the adjacent light emitting source of the pair is electrically coupled to conductive trace 1014. It will be understood that the relationship between the anode and cathode and the electrical trace to which it is coupled can be switched, so long as the appropriate polarity electrical current is applied to energize the light emitting sources so that they emit light. If the conductive traces are energized with an alternating current (AC), the anodes and cathodes of successive pairs of light emitting sources 1016 will preferably alternate in polarity in regard to their connection to conductive traces 1022 and 1014. The light emitting sources 1016 connected in one polarity are thus energized during the positive portion of the AC waveform, and those connected in the opposite polarity are energized during the negative portion of the AC waveform.

The two light emitting sources are connected in series using a flywire 1024 that extends between the anode of one of the pair of light emitting sources and the cathode of the other. Alternatively, it would be possible to directly connect flywire 1024 between one of the light emitting sources and the other conductive trace that it is not adhesively bonded to, so that the two light emitting sources are connected in parallel rather than in series. Other techniques for mounting the light emitting sources to the conductive traces can be used to eliminate the need for flywire 1024, for example, by directly connecting terminals (not shown) disposed at each side of the light emitting sources to the respective conductive traces.

A droplet 1026 of a flexible epoxy or other polymer may be applied over each pair of light emitting sources 1016 to protect them and flywire 1024. This droplet is optically transparent or translucent. Further, the surface of the flexible patch facing inwardly toward the treatment site may be coated with a relatively thin layer 1028 of silicone to insulate the entire assembly and provide protection to conductive traces 1012 and 1014 in those areas between droplets 1026. It may be desirable that this thin layer and the droplet applied over each light emitting source 1016 have an index of refraction that is generally matched to that of the subject's skin at the treatment site to which light therapy is to be administered by light emitting sources 1016. The maximum thickness of the flexible patch may, for example, be less than 1.0 millimeters, which may insure the substantial flexibility of the patch.

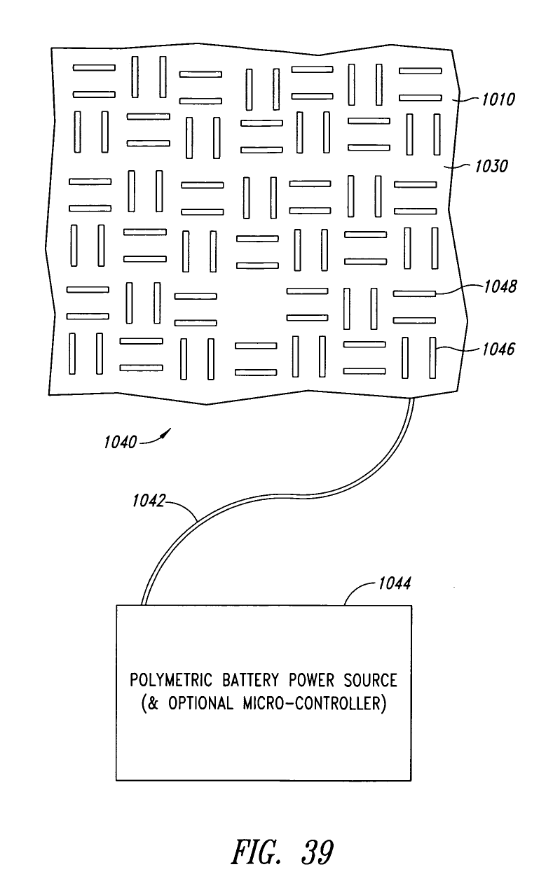

FIG. 39 shows a flexible patch 1040 fabricated using flexible substrate 1010. The light emitting sources 1016 mounted on the inwardly facing surface of flexible patch 1040 are disposed on the undersurface of the flexible substrate and thus do not show in this Figure.

To facilitate the flexible patch 1040 to fully conform to non-planar irregular surfaces on a subject's body, the flexible patch includes a plurality of openings 1048 and openings 1046 that extend through the flexible substrate and thin layer 1028. The openings or portions thereof may be orthogonally arranged with respect to the openings 1046, to provide stress relief about the at least two axes. Each of these openings also comprise open passages through which air and moisture are readily conveyed when flexible patch 1040 is applied to the treatment site on the subject's body. By providing such passages, irritation and heat buildup at the treatment site covered by flexible patch 1040 are minimized. Perspiration readily passes through these passages comprising openings 1048 and openings 1046 so that the subject is more comfortable during an extended period of light therapy provided by the flexible patch and to ensure that the patch remains adherently attached to the treatment site.

As shown in FIG. 39, a polymeric battery power source 1044 is coupled to the flexible patch through leads 1042. This power source provides the electrical current that energizes each of the light emitting sources mounted on the undersurface of flexible patch 1040. Optionally, polymeric battery power source 1044 includes a microcontroller. The purpose of the microcontroller is discussed below. A polymeric battery may more readily conform to the subject's body and be more comfortably carried than a rigid battery source, being flexible and adhesively attached to the subject's body. However, it is also contemplated that more conventional types of batteries may instead be used for providing electrical current to energize the light emitting sources used on flexible patch 1040. Clearly, many types of battery packs could be employed to provide the electrical current needed to energize the light emitting sources. It is also contemplated that the polymeric battery (or other type of battery power source that is used) be rechargeable to facilitate use of the flexible patch for an extended period of time by enabling the subject to repetitively recharge the power source as it becomes exhausted.

Assuming that the flexible substrate is optically transparent or at least partially translucent, the outer surface of flexible patch 1040 may optionally be coated with a reflective layer 1030. This reflective layer 1030 will reflect at least some of the light emitted by the light emitting sources back toward the treatment site, thereby increasing the efficiency with which light therapy is administered by the flexible patch.

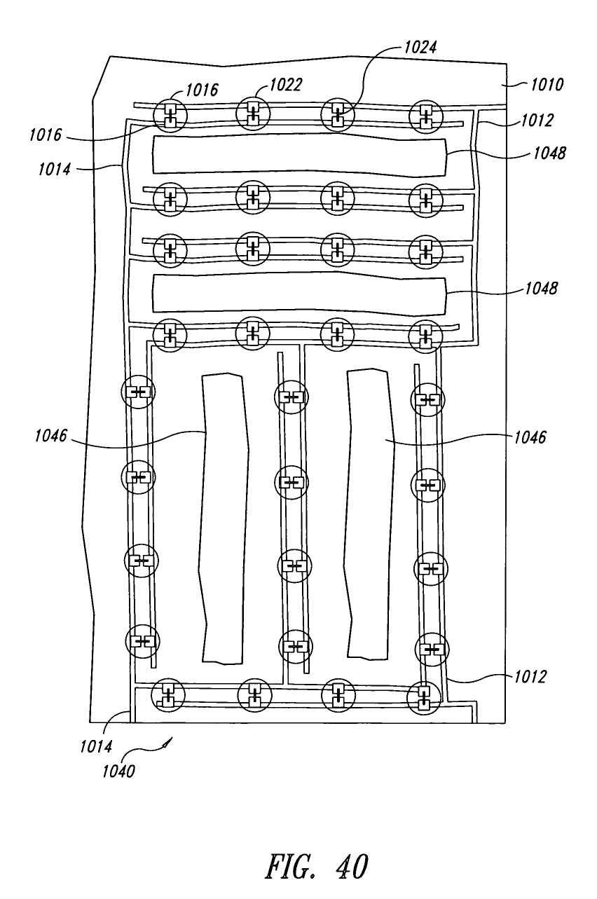

With reference to FIG. 40, a portion of flexible patch 1040 is enlarged, showing its undersurface and part of the electrical circuit comprising flexible traces 1012 and 1014. It will be noted in this Figure that conductive traces 1012 and 1014 are interspersed with openings 1048 and openings 1046 on the undersurface of the flexible patch; the light emitting sources 1016 thus comprise an array that is spaced apart over the remaining portion of the undersurface. While a simple pattern of the light emitting sources 1016, openings 1048, and openings 1046 is illustrated in FIG. 40, it will be apparent that many other configurations and patterns for electrical circuits comprising flexible traces 1012 and 1014 on which the light emitting sources 1016 are mounted interspersed with horizontal openings 1048 and vertical openings 1046 can alternatively be provided on the undersurface of the flexible substrate.

It should be noted that a plurality of separately controlled electrical circuits can be provided using conductive traces 1012 and 1014 so that distinct and separate groups of light emitting sources 1016 are defined on the undersurface of flexible patch 1040.

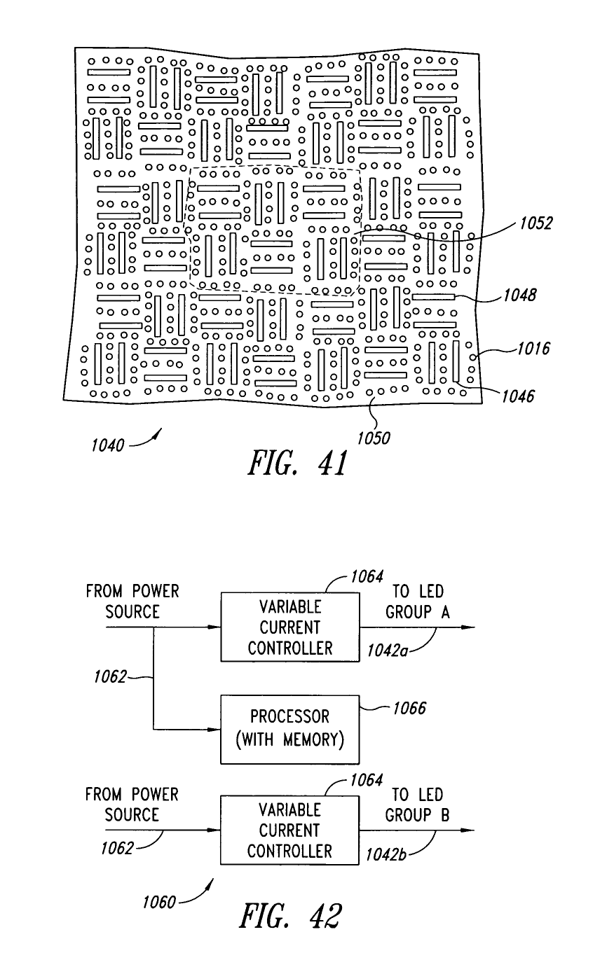

FIG. 41 illustrates a simple example in which a central group 1052 of light emitting sources 1016 is defined (encompassed by the dash line). Surrounding central group 1052 is a peripheral group 1050 of the light emitting sources 1016 that are separately controlled. An advantage of this simple configuration is that it provides an option to independently control the electrical current supplied to each different group to control the light intensity produced by the light emitting sources 1016 in each group. Thus, for example, central group 1052 can be energized longer or with a greater current, compared to that supplied to peripheral group 1050, to increase the intensity and/or the duration of the light produced by the central group of light emitting sources 1016. By increasing the light output of central group 1052, a more effective treatment of a tumor can be achieved, since the tumor is relatively thicker in its central part, where higher intensity and/or longer duration light therapy should be administered, and thinner around its periphery, where relatively lower intensity and/or shorter duration light therapy should be administered. It will be apparent that additional groups of light emitting sources 1016 can be configured and separately controlled to provide substantially more complex patterns to achieve other desired light distribution and control regions over the undersurface of flexible patch 1040 as necessary to meet the desired requirements for varying the light intensity over these portions of the treatment site. Also, the shape of any portion of a given group of light emitting sources 1016 on the undersurface of the flexible patch can be made substantially different than illustrated in FIG. 41 and might be, for example, “L-shaped,” oval-shaped, etc.

FIG. 42 illustrates functional components of a microcontroller circuit 1060 for use in selectively controlling the electrical currents supplied to each group of LEDs or other light emitting sources 1016. Lines 1062 convey the electrical power from the power source to a variable current controller 1064 and to a processor 1066. Preferably, processor 1066 comprises a simple microcontroller that includes both random access memory (RAM) and read only memory (ROM). Stored within the ROM is a simple operating system and a control application program comprising machine instructions that enable basic electrical current control functions to be implemented according to a time schedule and/or determining relative levels of electrical current to be supplied to each of a plurality of different groups of light emitting sources 1016. In the simple case illustrated in FIG. 42, the electrical current supplied to only two different groups of light emitting sources 1016 is controlled. However, it will be apparent that the electrical current supplied to additional groups of LEDs or other light emitting sources 1016 can be controlled to provide a desired light intensity and/or to determine a schedule for energizing each group. Variable current controller 1064 may comprise voltage controlled variable resistors, or pulse width modulation circuits for use in determining an amplitude or duration of the electrical current supplied to each group in response to a signal supplied by the processor. If pulse width modulation control is employed, the frequency of the pulses or a proportion of their time-on-versus-time-off will determine the light intensity of the light emitting sources 1016. The signal provided by the processor can also determine when and whether each group of light emitting sources 1016 is energized. Other control schemes can also be employed for modifying the light output of the light emitting sources 1016 in different areas of the undersurface of the flexible patch.

Provision of the horizontal and vertical openings can be provided in the patches described above. The conformal flexible light emitting patch 348 of FIG. 13 and its relatively thin cross section enable the conformal flexible light emitting patch 348 to deform and readily conform to the non-planar shape of the treatment site so that the conformal flexible light emitting patch 348 molds closely to the underlying surface of skin and molds smoothly over any non-planar areas such as joint. Since each of the light emitting sources are thus disposed immediately adjacent the treatment site, against the surface of the subject's skin, the light emitted thereby is readily able to penetrate through the cutaneous layer and reach subdermal portions to render PDT (or other light therapy).

In this example, the electrical current supplied to the central group of light sources of the flexible patch that overlie the thickest portion of the treatment site should be controlled to provide the maximum intensity and/or duration of light therapy administered thereto. The electrical current supplied to the peripheral group of the light emitting sources (e.g., the emitting sources 1016) can be lower than that supplied to the group of light emitting sources 1016 at the center of the conformal flexible light emitting patch 348 and/or its duration can be substantially shorter around the edges. By controlling the light intensity or duration of light therapy applied to the treatment site in this manner, a more effective treatment is achieved and the normal tissue does not receive an unnecessary exposure to higher intensity light and/or the length of exposure to the light required to treat the central portion of the treatment area.

Easier to administer therapy systems by providing more precise positioning methods and provide better access to posterior surfaces of the joint, and that more accurately deliver therapy to the desire location and/or joint. Other advantages include the ability to deliver therapy to novel therapy targets, including the synovium.

The advantages may include accurate location of therapy with little or no training required, precise positioning of the joint for therapy, novel therapy target—synovial fluid, and access to posterior surface of the joint (particularly the knee) for treating synovial fluid.

Light therapy treatment systems may be useful for treating inflammation, pain, damaged, or destroyed tissue and other conditions associated with, for example, injured tissues certain diseases (e.g., arthritis, tendonitis, and the like). The light therapy treatment systems are operable to deliver one or more doses of high intensity light to the body part of interest. A dose can comprise a therapeutically effective amount of high intensity light to selectively inhibit the progression of at least one condition associated with a disease, such as arthritis. Among the at least one condition examples include, without limitation, discomfort, pain, inflammation, tissue damage or destruction. In some embodiments, the therapeutically effective amount of light substantially prevents or reverses the progression of at least one condition associated with the disease. For example, light has been shown to promote cell growth (e.g., cell proliferation), aid in regeneration of tissue, aid in curing of tissue related diseases, reduce arthritic pain, reduce the rate of tissue damage, and the like. (see, e.g., Baranauska et al., “Laser treatment of experimentally induced chronic arthritis” Applied Surface Science, (561) pp. 154-55 (2000); Calatrava et al., “Histological and clinical responses of articular cartilage to low-level laser therapy: experimental study” Laser Med Sci. (12) pp. (1997) 117; Schultz, R., Krishnamurthy, S., Thelmo, W., Rodriguez, J. and Harvey G. (1985) Effects of varying intensities of laser energy on articular cartilage. Lasers Surg. Med. 5:577.) The light therapy treatment systems, in some embodiments, can deliver one or more doses of light to effectively treat pain, inflammation, discomfort, pain, legions, cartilage destruction or damage, or combinations thereof, and other known conditions. For example, the light therapy treatment systems can promote cell regeneration to counter (e.g., substantially offset the unwanted effects) at least one unwanted condition attributable to arthritis (or other similar diseases, conditions, symptoms, and the like). In some embodiments, the light therapy treatment systems can reduce or limit levels of pain or discomfort associated with arthritis. One skilled in the relevant arts can select and vary one or more of the operating parameters disclosed herein to treat a certain disease, condition, and/or symptom.

As noted above, the synovial fluid in the knee is contained in the synovial membrane and the bursae. They are roughly located 1) surrounding the patella, 2) along the midline of the joint, and 3) across the posterior surface of the knee joint. Existing devices are not able to deliver a therapeutic dose to all these areas with a precise therapy location. In addition, accessing the posterior surface of the knee (e.g., the knee 134 in FIG. 9) with a moveable brace or wrap is challenging because of the risk of restricting blood flow when the knee is bent while wearing a brace.

Other joints have similar anatomy consisting of synovial fluid contained in one or more sacs dispersed through the joint. Specific embodiments for other joints would have similar requirements.

All of the above U.S. patents, U.S. patent application publications, U.S. patent applications, foreign patents, foreign patent applications and non-patent publications referred to in this specification and/or listed in the Application Data Sheet, to include U.S. Pat. Nos. 6,958,498; 6,784,460; 6,661,167; 6,096,066; and 6,445,011; U.S. Publication Nos. 2005/0228260 and 2005/0085455; International Patent Application Nos. PCT/US2005/032851 and PCT/US01/44046; and U.S. Provisional Patent Application No. 60/728,556 are incorporated herein by reference in their entireties. Except as described herein, the embodiments, features, systems, devices, materials, methods and techniques described herein may, in some embodiments, be similar to any one or more of the embodiments, features, systems, devices, materials, methods and techniques described in the incorporated references. In addition, the embodiments, features, systems, devices, materials, methods and techniques described herein may, in certain embodiments, be applied to or used in connection with any one or more of the embodiments, features, systems, devices, materials, methods and techniques disclosed in the above-mentioned incorporated references.

The various methods and techniques described above provide a number of ways to carryout the invention. Of course, it is to be understood that not necessarily all objectives or advantages described may be achieved in accordance with any particular embodiment described herein. Certain embodiments may be suitable for treating specific disease or conditions. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments disclosed herein. For example, the various patches, light source arrays, panels, and circuitry can be incorporated into the various types of light delivery systems for providing light therapy on joints and other portions of a subject disclosed herein. Similarly, the various features and acts discussed above, as well as other known equivalents for each such feature or act, can be mixed and matched by one of ordinary skill in this art to perform methods in accordance with principles described herein. Additionally, the methods which are described and illustrated herein are not limited to the exact sequence of acts described, nor are they necessarily limited to the practice of all of the acts set forth. The methods may be altered for at home use or use a hospital or other healthcare facility. Other sequences of events or acts, or less than all of the events, or simultaneous occurrence of the events, may be utilized in practicing the embodiments of the invention.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. For example, the scale and size of the treatment systems can be adjusted to accommodate different body parts. For example, the treatment systems of FIGS. 1, 10A, and 13 can be sized for placement on the finger, toe, or other elongate body part. The materials, methods, ranges, and embodiments disclosed herein are given by way of example only and are not intended to limit the scope of the disclosure in any way. Accordingly, the invention is not limited except as by the appended claims.

1. A light therapy treatment system, comprising:
a power source;
a wearable positioning structure configured to receive a body part and to engage at least one anatomical feature of the body part so as to position itself with respect to the body part; and
a light emitting system coupled to the power source, the light emitting system being positioned relative to the positioning structure such that, when the body part is received by the positioning structure and the light emitting system receives energy from the power source, the light emitting system is positioned to deliver a therapeutically effective amount of light to a target site in the body part.

2. The light therapy treatment system of claim 1 wherein the wearable positioning structure has at least one anatomical feature locator corresponding in shape to the at least one anatomical feature such that the at least one anatomical feature locator engages the at least one anatomical feature to maintain alignment of the light emitting system with the target site.

3. The light therapy treatment system of claim 2 wherein the at least one anatomical feature locator comprises a conformable light patch dimensioned to be placed at least proximate a flexion crease in a posterior portion of the body part and a conformable light patch for placement on an anterior portion of the body part.

4. The light therapy treatment system of claim 1, further comprising:
a mounting system coupled to the positioning structure, the mounting system being movable between an open position for placing the body part in the positioning structure and a closed position for holding the body part securely in the positioning structure.

5. The light therapy treatment system of claim 1 wherein the light emitting system comprises one or more conformable light delivery patches configured to closely contact the body part, wherein the one or more conformable light delivery patches are capable of delivering the therapeutically effective amount of light to the target site.

6. The light therapy treatment system of claim 1 wherein the wearable positioning structure is aligned with respect to the body part when the body part comprises a joint that is at least partially bent.

7. The light therapy treatment system of claim 1 wherein the wearable positioning structure comprises a knee positioning structure and the body part comprises a knee, the light emitting system is configured to deliver the therapeutically effective amount of light energy to internal tissue of the knee.

8. The light therapy treatment system of claim 7 wherein the knee positioning structure is shaped and dimensioned to hold the knee at a knee flexion angle in a range of about 10 degrees to about 90 degrees.

9. The light therapy treatment system of claim 1, wherein the positioning structure comprises a lower elongate portion, an upper elongate portion, and an angled portion between the lower elongate portion and the upper elongate portion, the lower elongate portion is configured to receive at least a portion of a lower leg of the body part, the upper elongate portion is configured to receive at least a portion of a thigh of the body part, the angled portion is configured to receive at least a portion a knee joint of the body part and to define an angle of flexion of the knee.

10. The light therapy treatment system of claim 1 wherein the positioning structure is configured to guide the body part into physical contact with the light emitting system.

11. The light therapy treatment system of claim 1, further comprising:
a mounting system coupled to the positioning structure, the mounting system configured to hold the body part in engagement with the positioning structure, and the mounting system and the positioning structure form a brace that is sufficiently rigid to hold a joint of the body part in a predetermined range of positions.

12. The light therapy treatment system of claim 1 wherein the light therapy treatment system allows substantial joint movement during therapy.

13. The light therapy treatment system of claim 1 wherein the positioning structure has a preset configuration, the positioning structure in the preset configuration retains the body part in a treatment position that facilitates delivery of the light to the target site.

14. The light therapy treatment system of claim 1, further comprising:
a non-light penetrating energy delivery system positioned to selectively deliver a therapeutically synergistic amount of non-light energy to the target site.

15. The light therapy treatment system of claim 14 wherein the non-light energy comprises at least one of ultrasound energy, microwave energy, radiofrequency, mechanical pressure impulse energy, electromagnetic energy, and low level electrical currents.

16. The light therapy treatment system of claim 1 wherein the body part is a wrist, an elbow, a shoulder, a finger, a spine, a hip, an ankle, a foot, a hand, a jaw, or a toe.

17. The light therapy treatment system of claim 1 wherein the light emitting system is activated in response to signals from one or more pressure sensors positioned to engage the body part.

18. The light therapy treatment system of claim 1, further comprising:
a controller; and
at least one detector communicatively coupled to the controller, the at least one detector configured to measure a physiological indicator and to send a signal indicative of the physiological indicator, the controller configured to selectively command the light emitting system based at least in part on the signal from the at least one detector.

19. The light therapy treatment system of claim 1 wherein the physiological indicator is at least one of pressure, skin color, and temperature.

20. A treatment system for providing light therapy to a joint of a subject, comprising:
a joint brace comprising a main body configured to be placed adjacent the joint and an activatable light emitting system coupled to the main body, the light emitting system being capable of delivering a therapeutic amount of light energy to the joint when the main body is placed adjacent the joint.

21. The treatment system of claim 20 wherein the activatable light emitting system has a posterior light patch for delivering light to a posterior portion of the joint and an anterior light patch for delivering light to an anterior portion of the joint.

22. The treatment system claim 20 wherein the activatable light emitting system comprises one or more conformable light delivery patches configured to closely contact a portion of the subject at least near the joint, wherein the one or more conformable light delivery patches are capable of delivering the therapeutic amount of light to synovial tissue in the joint.

23. The treatment system of claim 20 wherein the joint brace further comprises:
a mounting system coupled to the main body, the mounting system being movable between an open position for placing the joint in the main body and a closed position for holding the joint in the main body.

24. The treatment system of claim 20 wherein the main body has a preset configuration for closely surrounding the joint.

25. The treatment system of claim 20 wherein the joint brace is a prophylactic brace.

26. The treatment system of claim 20 wherein the joint brace is a rehabilitative brace.

27. The treatment system of claim 20 wherein the joint brace is a functional brace.

28. The treatment system of claim 20, further comprising:
a non-light energy delivery system coupled to the joint brace, the non-light energy delivery system positioned relative to the main body such that the non-light energy delivery system is capable of selectively delivering a therapeutic effective amount of non-light energy to the joint.

29. A treatment system for providing therapy to a treatment site of a subject, comprising:
a wearable main body configured to be placed at least proximate the treatment site;
an activatable light emitting system coupled to the main body, the light emitting system being capable of delivering a therapeutic amount of light energy to the treatment site; and
an activatable non-light penetrating energy system coupled to the main body, the activatable non-light penetrating energy system being capable of delivering a therapeutic amount of non-light energy to the treatment site.

30.-48. (canceled)

49. A treatment system for providing therapy to a joint of a subject, comprising:
a wearable main body configured to be placed at least in proximity to the joint; and
an activatable light output system coupled to the main body, the light output system operable for delivering a therapeutically effective amount of light, the therapeutically effective amount comprising a sufficient amount of light, for a sufficient amount of time, to substantially inhibit progression of at least one condition associated with arthritis in the joint.

50. The system of claim 49 wherein the therapeutically effective amount of light substantially prevents the progression of the arthritis.

51. The system of claim 49 wherein the activatable light output system is capable of delivering light at an energy level equal to or greater than about 40 J/cm².

52. The system of claim 49 wherein the activatable light output system is capable of delivering light at an energy level equal to or greater than about 50 J/cm².

53. The treatment system of claim 49, further comprising:
a knee brace that includes the wearable main body and a mounting system for coupling the wearable main body to the joint.

54. A light therapy device, comprising:
a conformable light therapy patch comprising a substrate sufficiently flexible to conform to a non-planar portion of a subject that is to receive light therapy, a plurality of light emitting sources coupled to the substrate;
at least one circuit electrically coupling at least some of the light emitting sources; and
a structure configured to support the conformable light therapy patch while accommodating a joint of a subject that is to receive light therapy.

55.-73. (canceled)
